(19) United States
(12) Patent Application Publication
(10) Pub. No.: US 2024/0316069 A1
(43) Pub. Date: Sep. 26, 2024

(54) METHODS OF TREATING MULTIPLE SCLEROSIS

(71) Applicants: ASCENTAGE PHARMA (SUZHOU) CO., LTD., Suzhou, Jiangsu (CN); ASCENTAGE PHARMA GROUP CORP LIMITED, Hong Kong (CN)

(72) Inventors: Saijie ZHU, Suzhou (CN); Douglas D. FANG, Suzhou (CN); Yifan ZHAI, Suzhou (CN); Dajun YANG, Suzhou (CN)

(21) Appl. No.: 18/269,301

(22) PCT Filed: Dec. 28, 2021
(86) PCT No.: PCT/CN2021/141879

§ 371 Date: Jun. 23, 2023

(30) Foreign Application Priority Data
Dec. 28, 2020 (WO) PCTCN2020140197

(51) Int. Cl. A61K 31/635; A61K 31/496; A61P 25/28

(52) U.S. Cl. CPC A61K 31/635; A61K 31/496; A61P 25/28

(57) ABSTRACT

Disclosed are methods of treating multiple sclerosis. The method comprises: administering an effective amount of a compound represented by Formula I below, a pharmaceutically acceptable salt or solvate thereof to a subject in need thereof.

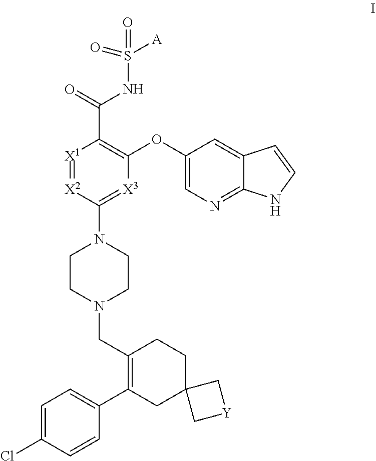

### FIELD OF THE INVENTION

The present disclosure relates to methods of treating multiple sclerosis (MS).

### BACKGROUND OF THE INVENTION

An autoimmune disease is a condition that results from a pathological immune reaction against an individual's own tissues. Examples of autoimmune diseases, in which the immune system attacks otherwise healthy tissue include multiple sclerosis (MS) and the like.

Multiple sclerosis (MS) is a debilitating disease of the central nervous system (CNS) in which the body's own immune system attacks the white matter of the brain and spinal cord. This triggers inflammation-induced damage to the CNS protective myelin layer causing demyelination. Loss of myelin exposes neurons to further attack leading to formation of multiple sclerotic lesions. This damage disrupts the ability of parts of the nervous system to communicate, resulting in a wide range of problems including fatigue, blurred vision, cognitive impairment, and spasticity.

MS takes several forms, with new symptoms either occurring in isolated attacks (relapsing forms) or building up over time (progressive forms).

There is at present no known cure for multiple sclerosis. Current treatments attempt to improve function after an attack and/or prevent subsequent attacks.

Medications used to treat MS, while modestly effective, can have adverse effects and be poorly tolerated.

There is ongoing research looking for more effective, convenient, and tolerable treatments for relapsing-remitting MS.

### BRIEF SUMMARY OF THE PRESENT INVENTION

It has now been discovered that a Bcl-2 inhibitor is effective to treat multiple sclerosis. In particular embodiments, the Bcl-2 inhibitor is a compound represented by any one of Formula I-IV below, the pharmaceutically acceptable salts or solvates, e.g., hydrates, thereof, collectively referred to herein as “Compounds of the Disclosure”.

In one aspect, the present disclosure provides a method of treating multiple sclerosis, which comprises administering an effective amount of Compounds of the Disclosure to a subject in need thereof.

In another aspect, the present disclosure provides a method of reducing inflammation, which comprises administering an effective amount of Compounds of the Disclosure to a subject in need thereof.

In another aspect, the present disclosure provides a method of reducing the level of leukocytes, T lymphocytes and B lymphocytes in the PBMC (peripheral blood mononuclear cells) in a subject, comprising administering to the subject an effective amount of Compounds of the Disclosure, wherein the subject suffers from multiple sclerosis.

In another aspect, the present disclosure provides a method of inducing an apoptosis-resistant cell to undergo apoptosis, the cell being associated with multiple sclerosis, the method comprising: sensitizing the apoptosis-resistant cell to apoptosis stimuli by reacting the cell with at least one Compounds of the Disclosure, so that the cell undergoes apoptosis at a site of multiple sclerosis.

In one embodiment, the present disclosure provides a method of inducing apoptosis in an apoptosis-resistant cell, the cell being associated with multiple sclerosis, the method comprising: sensitizing the apoptosis-resistant cell to apoptosis stimuli by treating the cell with at least one Compounds of the Disclosure, so that the cell undergoes apoptosis at a site of multiple sclerosis.

In another aspect, the present disclosure provides a therapeutic agent for multiple sclerosis, comprising Compounds of the Disclosure.

In another aspect, the present disclosure provides a pharmaceutical composition for treating multiple sclerosis, wherein the pharmaceutical composition comprises Compounds of the Disclosure and one or more pharmaceutically acceptable carriers.

In another aspect, the present disclosure provides use of Compounds of the Disclosure as a medicament of multiple sclerosis.

In another aspect, the present disclosure provides Compounds of the Disclosure for use in treating multiple sclerosis in a subject.

In another aspect, the present disclosure provides use of Compounds of the Disclosure in the manufacture of a medicament for treating multiple sclerosis.

In another aspect, the present disclosure provides a kit for treating multiple sclerosis, wherein the kit comprising Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

### BRIEF DESCRIPTION OF THE DRAWING

FIG. 1 shows Compound 4 reduced the clinical scores in MOG35-55 induced EAE model in C57BL/6 mice.

FIG. 2 shows Compound 4 reduced the LFB staining scores in MOG35-55 induced EAE model in C57BL/6 mice.

FIG. 3 shows Compound 4 reduced the H&E staining scores in MOG35-55 induced EAE model in C57BL/6 mice.

FIG. 4 to FIG. 9 show Compound 4 reduced the number of leukocytes, T lymphocytes, B lymphocytes, CD4+ T lymphocytes, CD8+ T lymphocytes, and CD4-CD8− T lymphocytes in the PBMC in MOG35-55 induced EAE model in C57BL/6 mice.

### DETAILED DESCRIPTION OF THE INVENTION

Compounds of the Disclosure reduce the levels of leukocytes, T lymphocytes and B lymphocytes in the PBMC in a subject. In view of this property, Compounds of the Disclosure are useful for treating diseases, e.g., MS, in a subject.

In one embodiment, the present disclosure provides a method of treating multiple sclerosis, comprising administering to a subject in need thereof a compound represented by Formula I below, a pharmaceutically acceptable salt or solvate thereof:

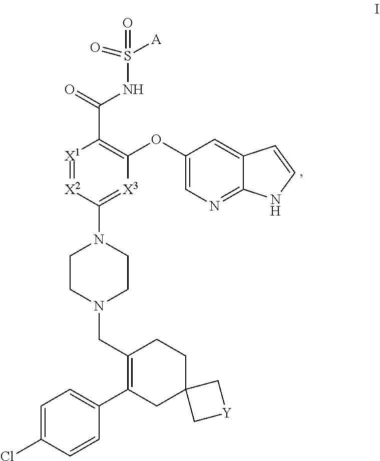

wherein:
A is

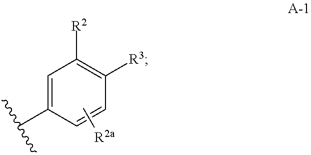

X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;

R^{4b} is selected from the group consisting of hydrogen and C₁₋₄ alkyl; and

Y selected from the group consisting of —CH₂— and —O—.

In another embodiment, Compounds of the Disclosure are compounds having Formula II:

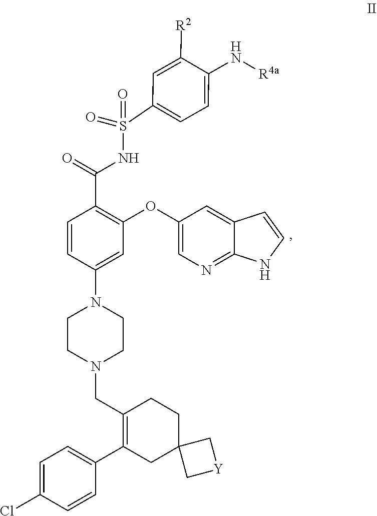

or a pharmaceutically acceptable salt or solvate thereof, wherein Y selected from the group consisting of —CH₂— and —O—, and R² and R^{4a} are as defined in connection with Formula I.

In another embodiment, Compounds of the Disclosure are compounds having Formula III:

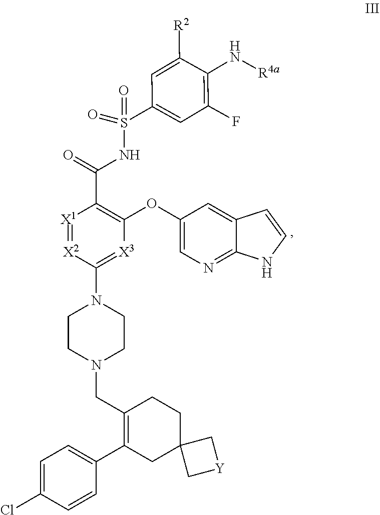

or a pharmaceutically acceptable salt or solvate thereof, wherein Y selected from the group consisting of —CH₂— and —O—, and X¹, X², X³, R², and R^{4a} are as defined in connection with Formula I.

In another embodiment, Compounds of the Disclosure are compounds having Formula I, or III, or a pharmaceutically acceptable salt or solvate thereof, wherein X¹, X², and X³ are each —CH═.

In another embodiment, Compounds of the Disclosure are compounds having Formula I, or III, or a pharmaceutically acceptable salt or solvate thereof, wherein X¹ is —CF═, and X² and X³ are each —CH═.

In another embodiment, Compounds of the Disclosure are compounds having Formula I, or III, or a pharmaceutically acceptable salt or solvate thereof, wherein X¹ and X³ are each —CH═, and X² is —CF═.

In another embodiment, Compounds of the Disclosure are compounds having Formula I, or III, or a pharmaceutically acceptable salt or solvate thereof, wherein X¹ and X² are each —CH═, and X³ is —CF═.

In another embodiment, Compounds of the Disclosure are compounds having Formula I, or III, or a pharmaceutically acceptable salt or solvate thereof, wherein X¹ is —N═, and X² and X³ are each —CH═.

In another embodiment, Compounds of the Disclosure are compounds having Formula I, or III, or a pharmaceutically acceptable salt or solvate thereof, wherein X¹ and X³ are each —CH═, and X² is —N═.

In another embodiment, Compounds of the Disclosure are compounds having Formula I, or III, or a pharmaceutically acceptable salt or solvate thereof, wherein X¹ and X² are each —CH═, and X³ is —N═.

In another embodiment, Compounds of the Disclosure are compounds having any one of Formulae I, II or III, or a pharmaceutically acceptable salt or solvate thereof, wherein Y is —O—.

In another embodiment, Compounds of the Disclosure are compounds having any one of Formulae I, II or III, or a pharmaceutically acceptable salt or solvate thereof, wherein Y is —CH₂—.

In another embodiment, Compounds of the Disclosure are compounds having any one of Formulae I, II or III, or a pharmaceutically acceptable salt or solvate thereof, wherein R² is —NO₂.

In another embodiment, Compounds of the Disclosure are compounds having any one of Formulae II, or a pharmaceutically acceptable salt or solvate thereof, wherein R^{4a} is selected from the group consisting of:

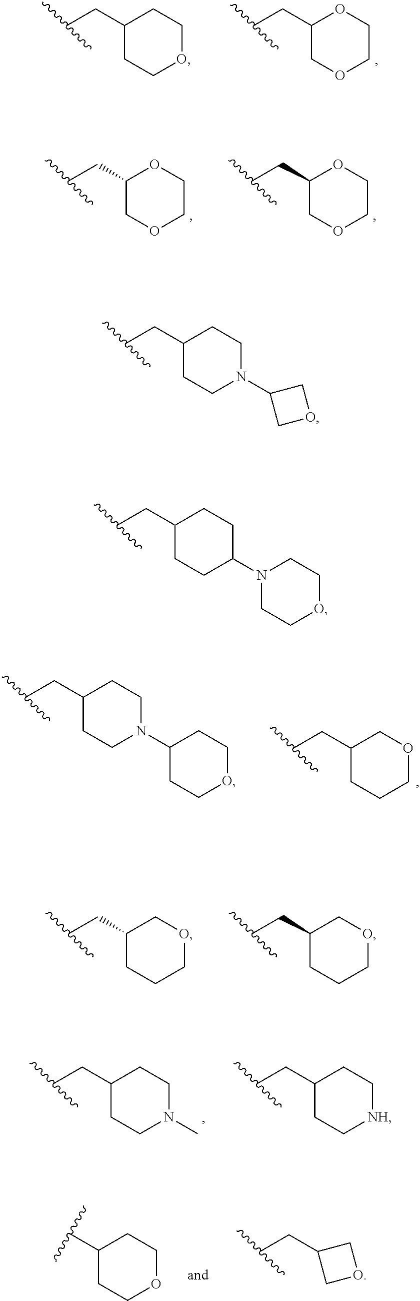

In another embodiment, Compounds of the Disclosure are compounds having any one of Formulae I, or III, or a pharmaceutically acceptable salt or solvate thereof, wherein R^{4a} is selected from the group consisting of:

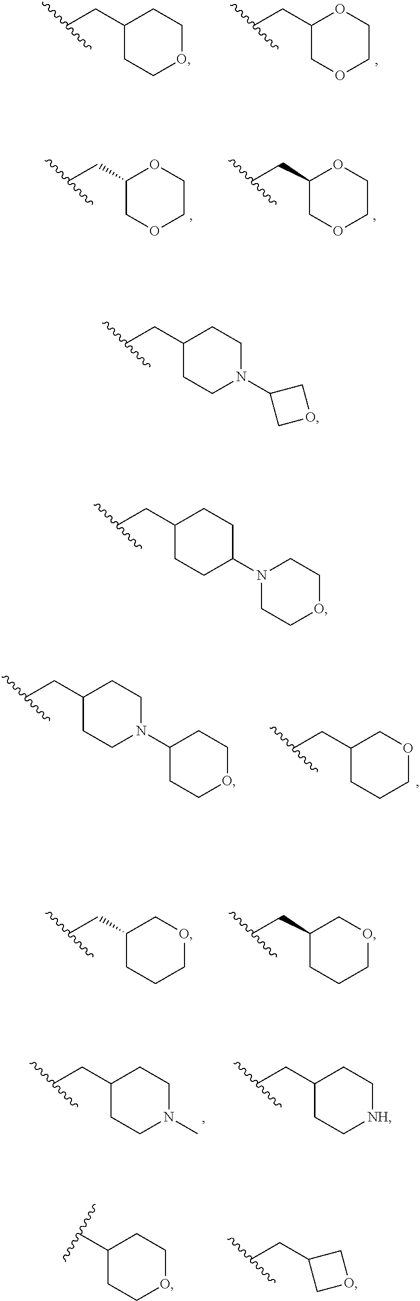

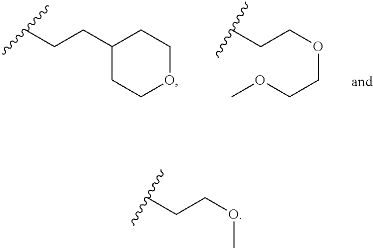

In another embodiment, Compounds of the Disclosure are compounds having Formula IV;

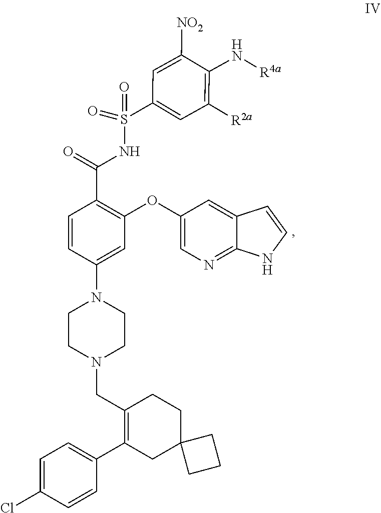

or a pharmaceutically acceptable salt or solvate thereof, wherein R^{2a} is hydrogen or fluoro and R^{4a} is as defined in connection with Formula I.

In another embodiment, Compounds of the Disclosure are compounds having Formula IV, or a pharmaceutically acceptable salt or solvate thereof, wherein R^{4a} is selected from the group consisting of:

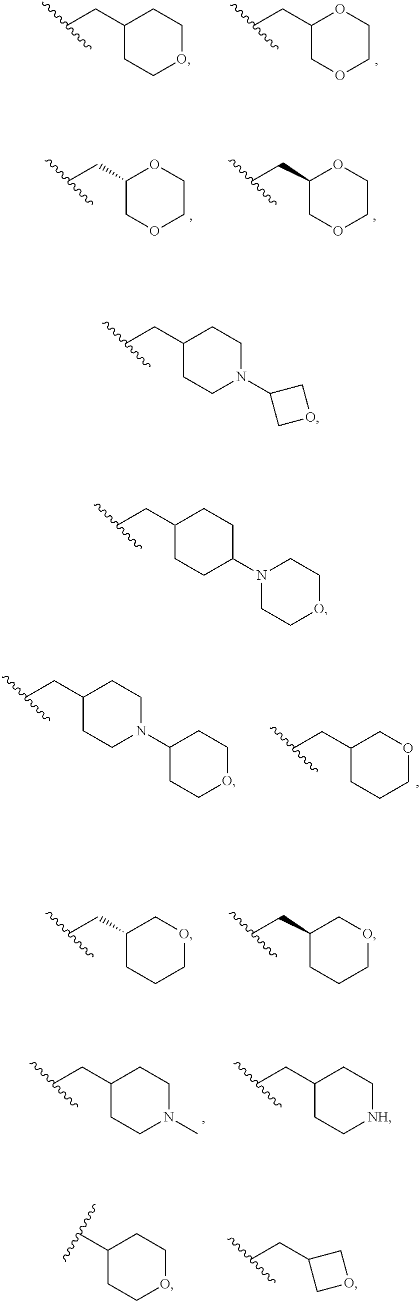

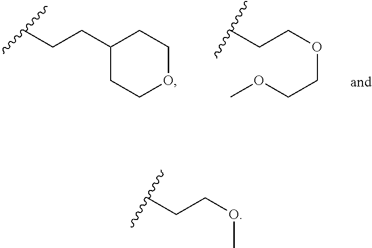

In another embodiment, Compounds of the Disclosure are compounds selected from one or more of the compounds of Table 1, or a pharmaceutically acceptable salt or solvate thereof.

| Cpd. | | |
|---|---|---|
| No. | Structure | Name |
| 1 | 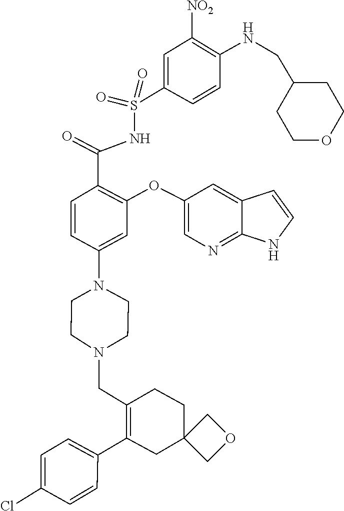 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(4- ((6-(4-chlorophenyl)-2-oxaspiro[3.5]non-6-en- 7-yl)methyl)piperazin-1-yl)-N-((3-nitro-4- (((tetrahydro-2H-pyran-4- yl)methyl)amino)phenyl)sulfonyl)benzamide |
| 2 | 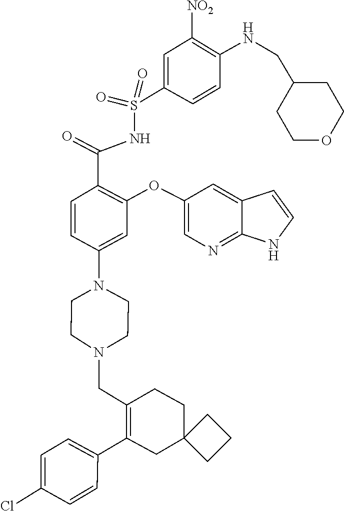 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)benzamide |
| 3 | 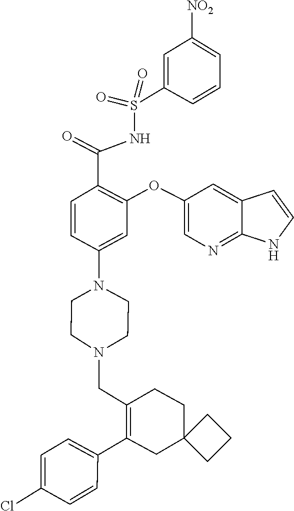 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3- nitrophenyl)sulfonyl)benzamide |
| 4 | 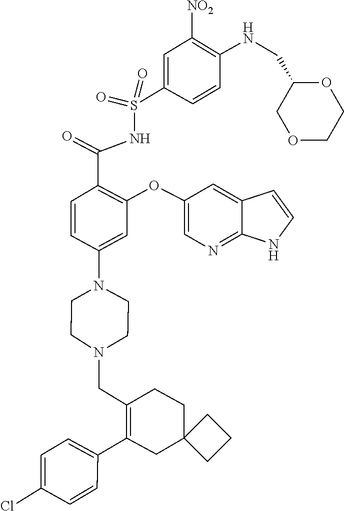 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 5 | 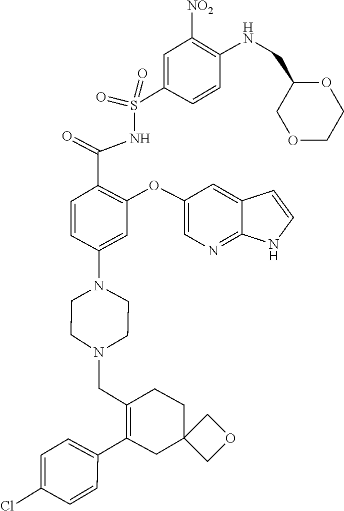 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4-chlorophenyl)- 2-oxaspiro[3.5]non-6-en-7- yl)methyl)piperazin-1-yl)benzamide |
| 6 | 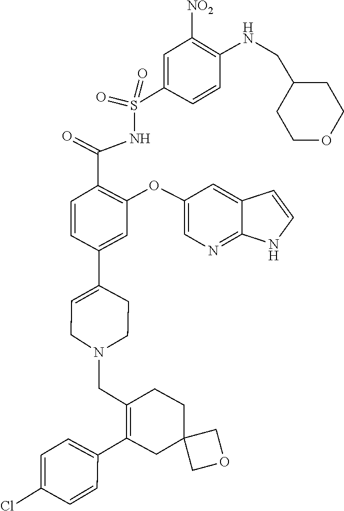 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(1- ((6-(4-chlorophenyl)-2-oxaspiro[3.5]non-6-en- 7-yl)methyl)-1,2,3,6-tetrahydropyridin-4-yl)- N-((3-nitro-4-(((tetrahydro-2H-pyran-4- yl)methyl)amino)phenyl)sulfonyl)benzamide |
| 7 | 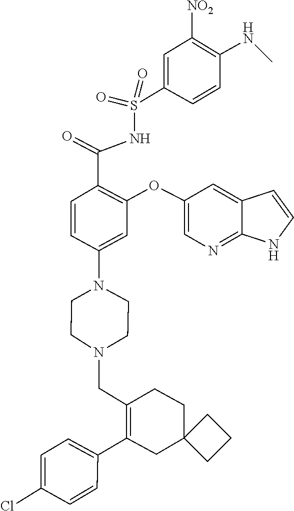 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4- (methylamino)-3- nitrophenyl)sulfonyl)benzamide |
| 8 | 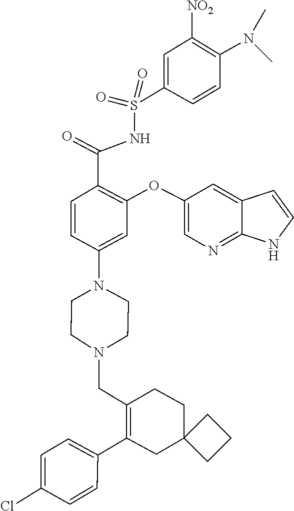 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4- (dimethylamino)-3- nitrophenyl)sulfonyl)benzamide |
| 9 | 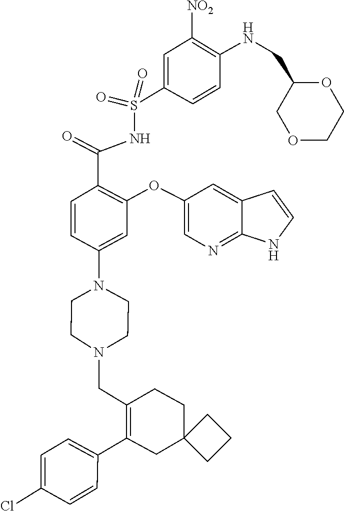 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 10 | 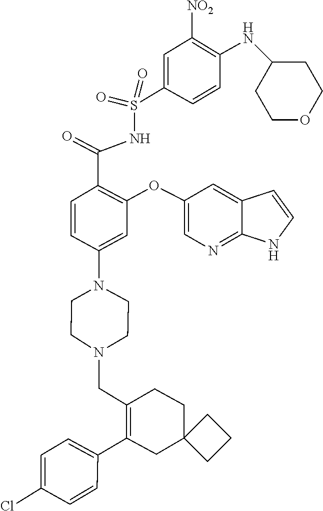 [SMILES: O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((tetrahydro-2H-pyran-4- yl)amino)phenyl)sulfonyl)benzamide |
| 11 | 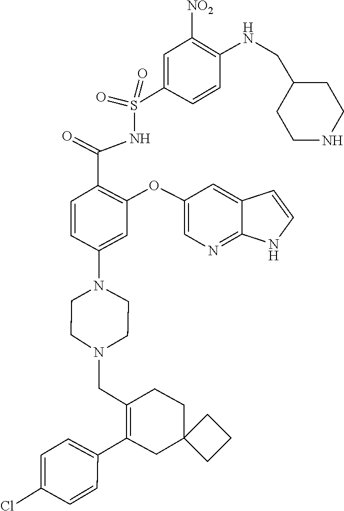 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((piperidin-4- ylmethyl)amino)phenyl)sulfonyl)benzamide |
| 12 | 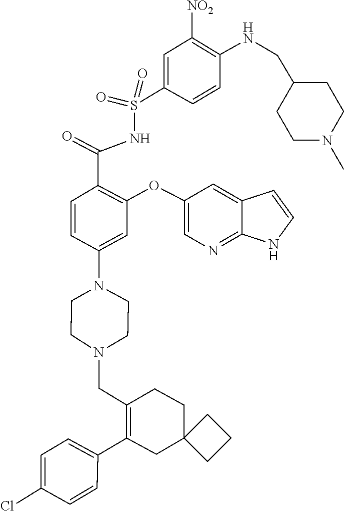 [SMILES: CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-(((1- methylpiperidin-4-yl)methyl)amino)-3- nitrophenyl)sulfonyl)benzamide |
| 13 | 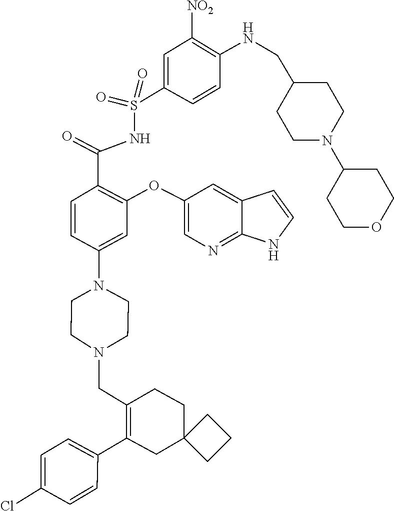 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4-(((1- (tetrahydro-2H-pyran-4-yl)piperidin-4- yl)methyl)amino)phenyl)sulfonyl)benzamide |
| 14 | 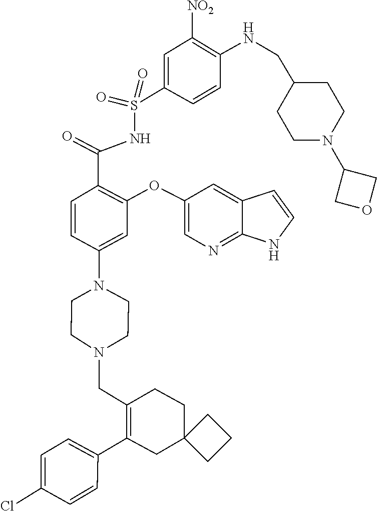 [SMILES: O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4-(((1- (oxetan-3-yl)piperidin-4- yl)methyl)amino)phenyl)sulfonyl)benzamide |
| 15 | 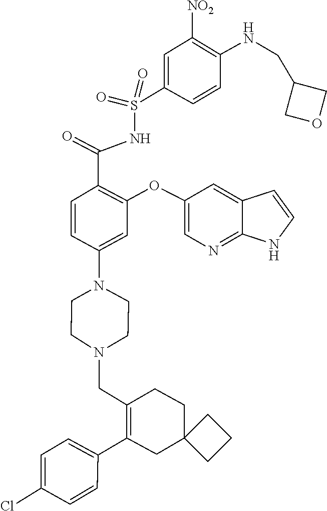 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((oxetan-3- ylmethyl)amino)phenyl)sulfonyl)benzamide |
| 16 | 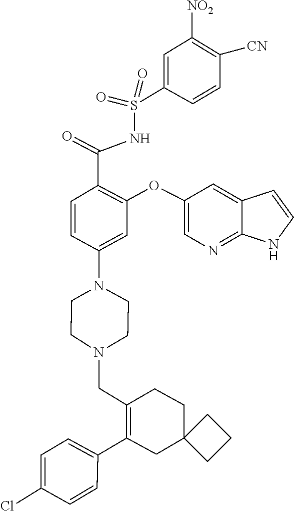 [SMILES: N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-cyano-3- nitrophenyl)sulfonyl)benzamide |
| 17 | 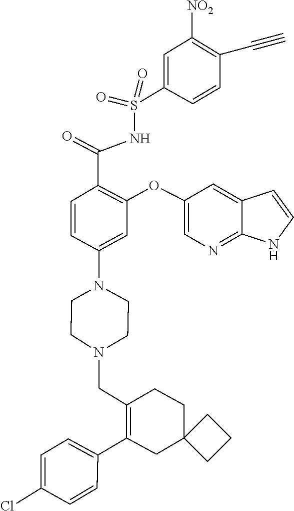 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-ethynyl-3- nitrophenyl)sulfonyl)benzamide |
| 18 | 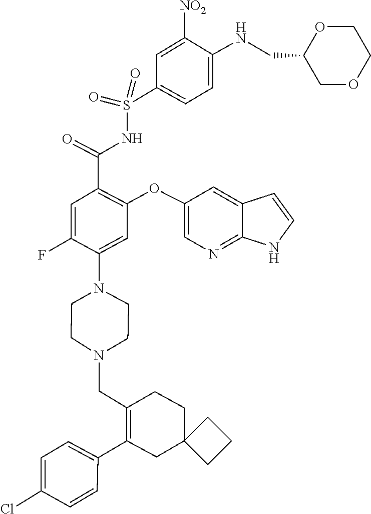 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-5-fluorobenzamide |
| 19 | 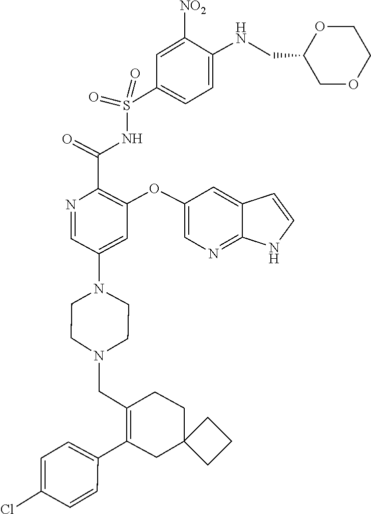 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-3-((1H-pyrrolo[2,3- b]pyridin-5-yl)oxy)-5-(4-((6-(4- chlorophenyl)spiro[3.5]non-6-en-7- yl)methyl)piperazin-1-yl)picolinamide |
| 20 | 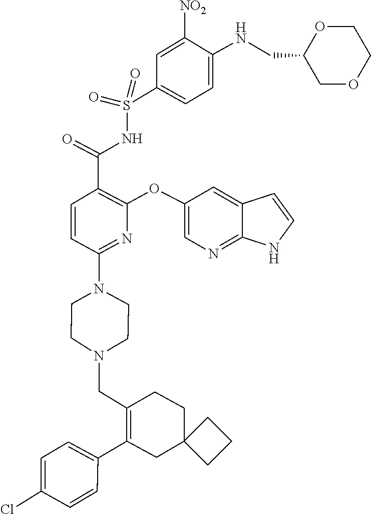 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-6-(4-((6-(4- chlorophenyl)spiro[3.5]non-6-en-7- yl)methyl)piperazin-1-yl)nicotinamide |
| 21 | 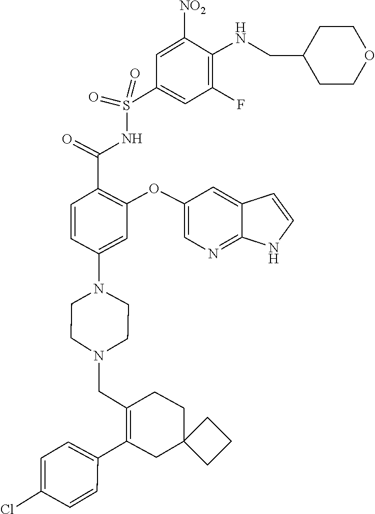 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-fluoro-5-nitro- 4-(((tetrahydro-2H-pyran-4- yl)methyl)amino)phenyl)sulfonyl)benzamide |
| 22 | 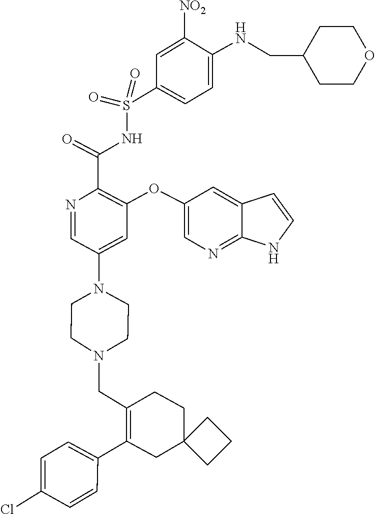 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1] | 3-((1H-pyrrolo[2,3-b]pyridin-5-yl)oxy)-5-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)picolinamide |
| 23 | 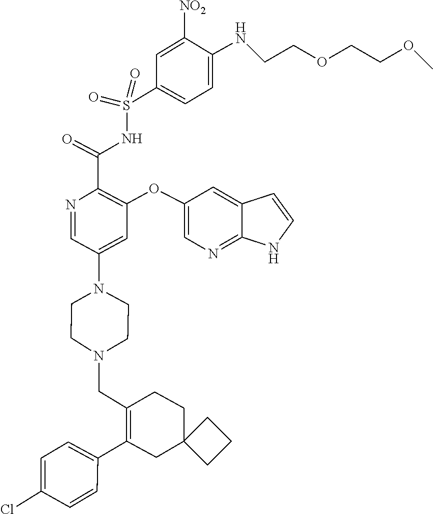 [SMILES: COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]] | 3-((1H-pyrrolo[2,3-b]pyridin-5-yl)oxy)-5-(4- ((6-(4-chlorophenyl)spiro[3.5]non-6-en-7- yl)methyl)piperazin-1-yl)-N-((4-((2-(2- methoxyethoxy)ethyl)amino)-3- nitrophenyl)sulfonyl)picolinamide |
| 24 | 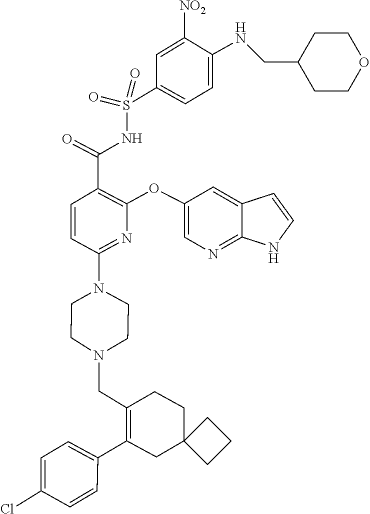 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1] | 2-((1H-pyrrolo[2,3-b]pyridin-5-yl)oxy)-6-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)nicotinamide |
| 25 | 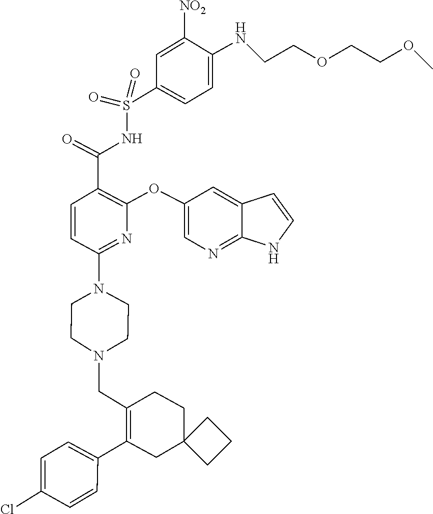 [SMILES: COCCOCCNc1ccc(S(=O)(=O)NC(=O)c2ccc(N3CCN(CC4=C(c5ccc(Cl)cc5)CC5(CCC5)CC4)CC3)nc2Oc2cnc3[nH]ccc3c2)cc1[N+](=O)[O-]] | 2-((1H-pyrrolo[2,3-b]pyridin-5-yl)oxy)-6-(4- ((6-(4-chlorophenyl)spiro[3.5]non-6-en-7- yl)methyl)piperazin-1-yl)-N-((4-((2-(2- methoxyethoxy)ethyl)amino)-3- nitrophenyl)sulfonyl)nicotinamide |
| 26 | 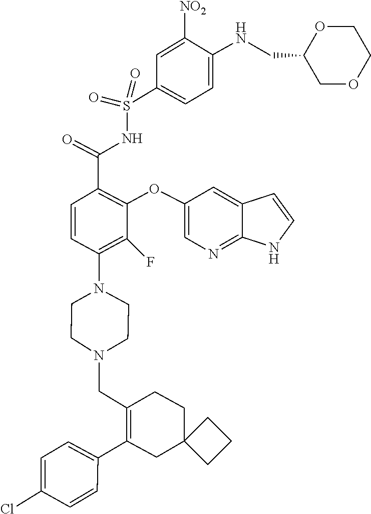 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-3-fluorobenzamide |
| 27 | 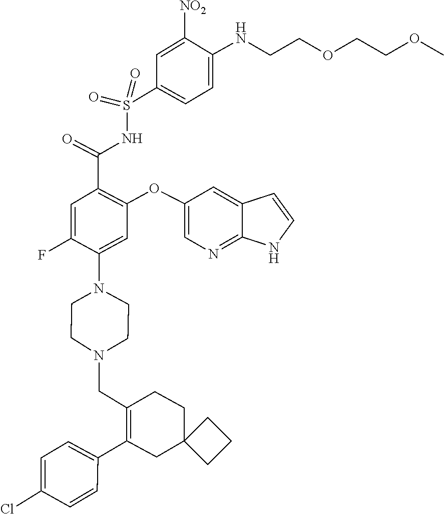 [SMILES: COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-5-fluoro-N-((4-((2- (2-methoxyethoxy)ethyl)amino)-3- nitrophenyl)sulfonyl)benzamide |
| 28 | 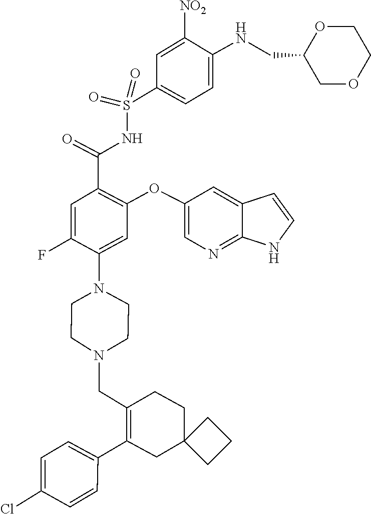 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-5-fluorobenzamide |
| 29 | 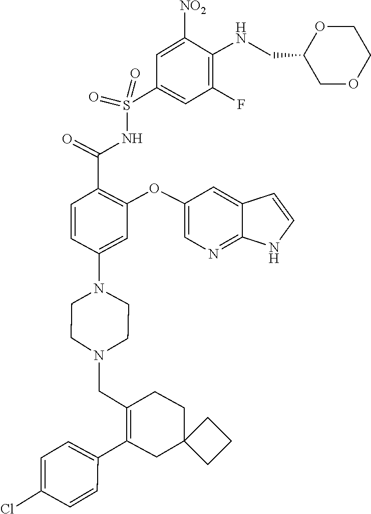 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- fluoro-5-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 30 | 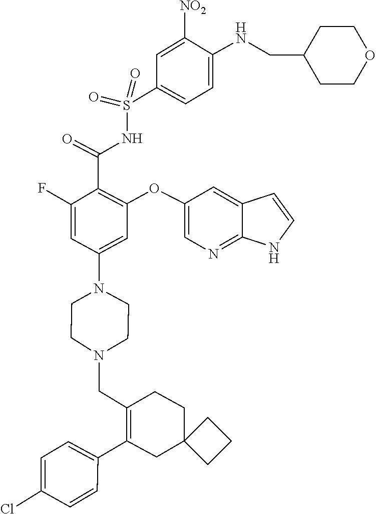 [SMILES: O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-6-fluoro-N-((3-nitro- 4-(((tetrahydro-2H-pyran-4- yl)methyl)amino)phenyl)sulfonyl)benzamide |
| 31 | 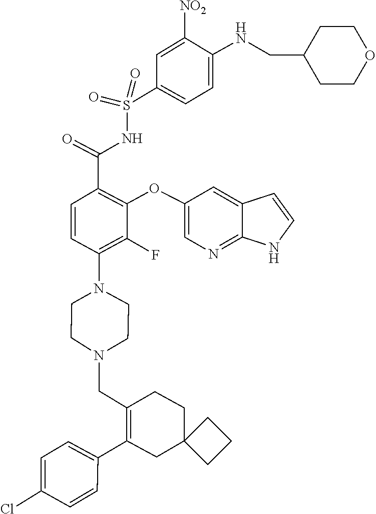 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-3-fluoro-N-((3-nitro- 4-(((tetrahydro-2H-pyran-4- yl)methyl)amino)phenyl)sulfonyl)benzamide |
| 32 | 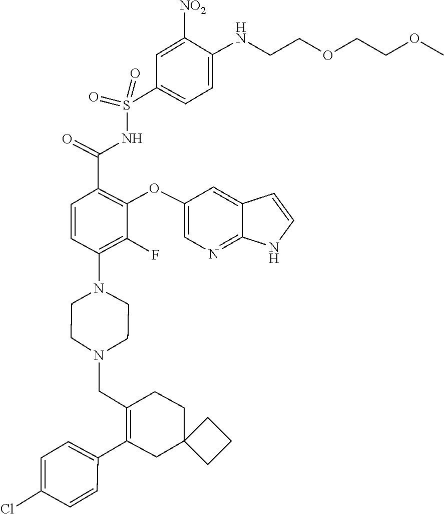 [SMILES: COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-3-fluoro-N-((4-((2- (2-methoxyethoxy)ethyl)amino)-3- nitrophenyl)sulfonyl)benzamide |
| 33 | 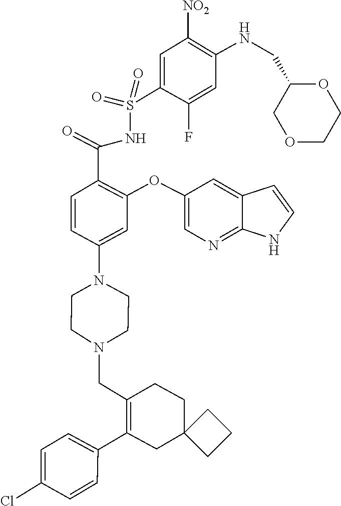 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-2- fluoro-5-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 34 | 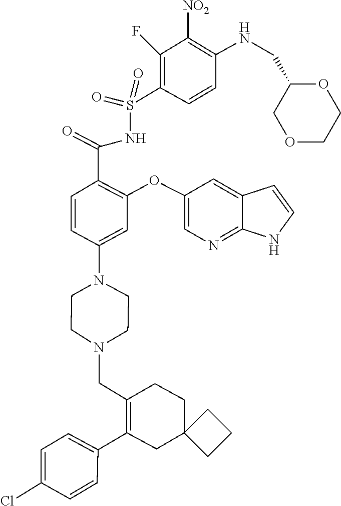 [SMILES: O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-2- fluoro-3-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 35 | 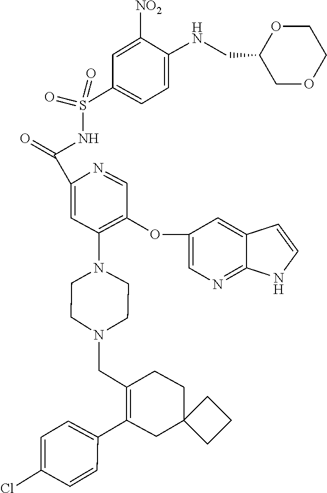 [SMILES: O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1] | (S)-N-((4-(((1,4-dioxan-2-yl)methyl)amino)-3- nitrophenyl)sulfonyl)-5-((1H-pyrrolo[2,3- b]pyridin-5-yl)oxy)-4-(4-((6-(4- chlorophenyl)spiro[3.5]non-6-en-7- yl)methyl)piperazin-1-yl)picolinamide |

In another embodiment, Compounds of the Disclosure are selected from the group consisting of:

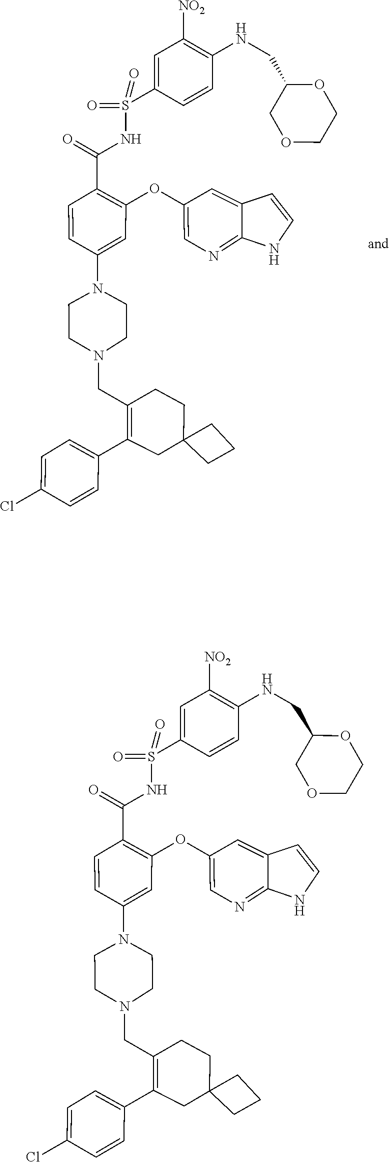 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or solvate thereof.

In another embodiment, Compounds of the Disclosure are selected from the group consisting of:

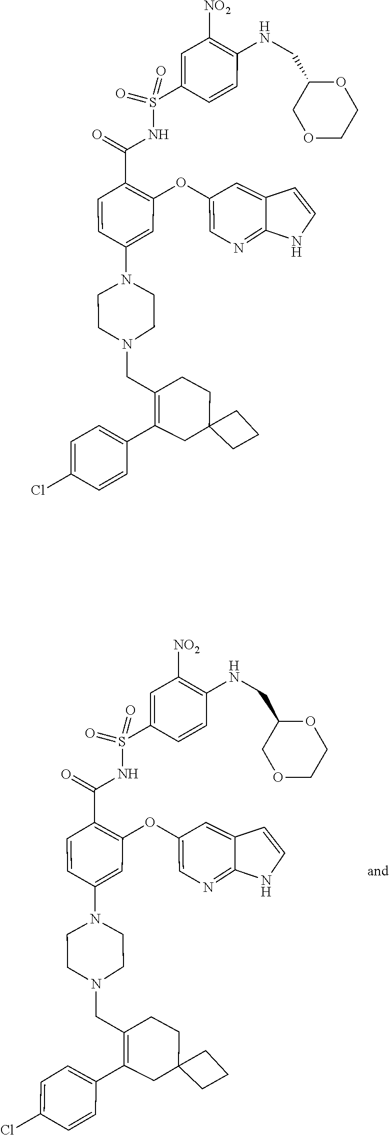 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

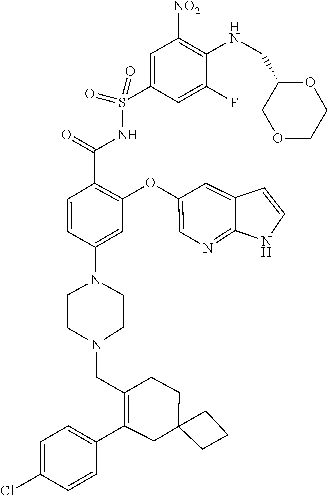 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or solvate thereof.

In another embodiment, a Compound of the Disclosure is Compound 4:

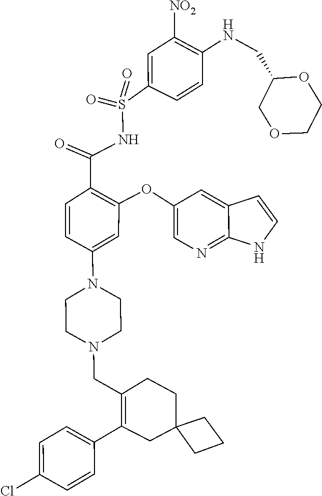 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or solvate thereof.

In another embodiment, a Compound of the Disclosure is:

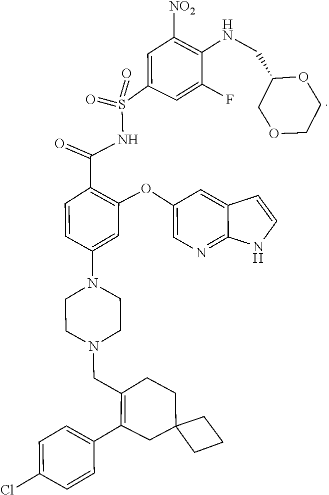 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or solvate thereof.

In one embodiment, the multiple sclerosis is relapsing-remitting multiple sclerosis, primary progressive multiple sclerosis or secondary progressive multiple sclerosis.

In one embodiment, the subject is a mammal.

In another embodiment, the subject is a mice or a human.

In one embodiment, Compounds of the Disclosure is administered in an amount of 0.0025 mg/kg to about 1.5 g/kg, e.g., about 0.0025 mg/kg to about 1 g/kg, about 0.0025 mg/kg to about 500 mg/kg, about 0.0025 mg/kg to about 250 mg/kg, about 0.0025 mg/kg to about 200 mg/kg, about 0.0025 mg/kg to about 100 mg/kg, about 0.0025 mg/kg to about 50 mg/kg, about 0.0025 mg/kg to about 10 mg/kg, to a subject in need thereof.

A unit dose may comprise from 0.01 mg to about 1.5 g of Compounds of the Disclosure, e.g., about 0.01 mg to about 1 g, about 0.01 mg to about 500 mg, about 0.01 mg to about 250 mg, about 0.01 mg to about 200 mg, about 0.01 mg to about 100 mg, about 0.01 mg to about 50 mg, about 0.01 mg to about 10 mg. The unit dose can be administered one or more times daily, e.g., as one or more tablets or capsules, each containing from about 0.01 mg to about 1.5 g of the compound, or an equivalent amount of a pharmaceutically acceptable salt or solvate thereof.

In certain embodiments, Compounds of the Disclosure is administered continuously for at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, least 50 days, at least 1 week, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In one embodiment, Compounds of the Disclosure is administered orally.

The present disclosure is further directed to a method of reducing inflammation in a subject in need thereof, the method comprises administering an effective amount of at least one Compounds of the Disclosure to the subject in need thereof.

In one embodiment, the inflammation is reduced in the periphery of the subject.

In one embodiment, the inflammation is reduced in the central nervous system of the subject.

In one embodiment, the inflammation is neuroinflammation.

In one embodiment, the neuroinflammation is caused by multiple sclerosis, such as relapsing-remitting multiple sclerosis, primary progressive multiple sclerosis, secondary progressive multiple sclerosis.

In one embodiment, the present disclosure provides a method of reducing the level of leukocytes, T lymphocytes and B lymphocytes in the PBMC in a subject, comprising administering to the subject Compounds of the Disclosure, wherein the subject suffers from multiple sclerosis.

In one embodiment, the present disclosure provides a method of inducing an apoptosis-resistant cell to undergo apoptosis, the cell being associated with multiple sclerosis, the method comprising: sensitizing the apoptosis-resistant cell to apoptosis stimuli by reacting the cell with at least one Compounds of the Disclosure, so that the cell undergoes apoptosis at a site of multiple sclerosis.

In one embodiment, the apoptosis-resistant cell is a T-cell, or a B cell.

In one embodiment, the T-cell is a CD45+CD3+ T-cell, CD45+CD3+CD4+ T-cell, CD45+CD3+CD8+ T-cell or CD45+CD3+CD4-CD8− T-cell.

In one embodiment, the B-cell is a CD45+CD3-B220+ B-cell.

In one embodiment, the site of multiple sclerosis is brain, myelin, intestinal mucosa, skin, or synovium.

In one embodiment, the site of multiple sclerosis is brain or myelin.

In one embodiment, the present disclosure provides a method of inducing apoptosis in an apoptosis-resistant cell, the cell being associated with multiple sclerosis, the method comprising: sensitizing the apoptosis-resistant cell to apoptosis stimuli by treating the cell with at least one Compounds of the Disclosure, so that the cell undergoes apoptosis at a site of multiple sclerosis.

In one embodiment, the present disclosure provides a therapeutic agent for multiple sclerosis, comprising Compounds of the Disclosure.

In another aspect, the present disclosure provides a pharmaceutical composition for treating multiple sclerosis, wherein the pharmaceutical composition comprises Compounds of the Disclosure and one or more pharmaceutically acceptable carriers.

In another embodiment, the present disclosure provides use of Compounds of the Disclosure as a medicament of multiple sclerosis.

In another embodiment, the present disclosure provides Compounds of the Disclosure for use in treating multiple sclerosis in a subject.

In another embodiment, the present disclosure provides use of Compounds of the Disclosure in the manufacture of a medicament for treating multiple sclerosis.

In another embodiment, the present disclosure provides a kit for treating multiple sclerosis, wherein the kit comprising Compounds of the Disclosure.

### Definitions

The terms “a” and “an” refer to one or more than one (i.e., at least one) of the grammatical object of the article. By way of example, “an element” means one element or more than one element.

The term “about”, as used herein, includes the recited number ±20%, 10%, 5%, or 1%. In certain embodiments, the term “about” is used to modify a numerical value above and below the stated value by a variance of 10%. Thus, “about 10” means 9 to 11. In certain embodiments, the term “about” is used to modify a numerical value above and below the stated value by a variance of 5%. In certain embodiments, the term “about” is used to modify a numerical value above and below the stated value by a variance of 1%.

It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof.

In one embodiment, the multiple sclerosis is, e.g., relapsing-remitting multiple sclerosis, primary progressive multiple sclerosis or secondary progressive multiple sclerosis.

The term “subject” is a mammal, including a human. Non-human animals subject include, for example, primates, mice, rats, cattle, sheep, goats, horses, canines, felines, and the like.

The term “halogen” refers to —Cl, —F, —Br, or —I.

The term “alkyl” as used by itself or as part of another group refers to the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In another embodiment, the alkyl group is a branched chain C₃₋₄ alkyl group. In another embodiment, the alkyl group is a straight or branched chain C₃₋₄ alkyl group. In another embodiment, the alkyl group is partially or completely deuterated, i.e., one or more hydrogen atoms of the alkyl group are replaced with deuterium atoms. Non-limiting exemplary C₁₋₄ alkyl groups include methyl, ethyl, propyl, isopropyl, butyl, sec-butyl, tert-butyl, and iso-butyl. Non-limiting exemplary C₁₋₄ groups include methyl, ethyl, propyl, isopropyl, and tert-butyl.

The term “optionally substituted alkyl” as used by itself or as part of another group refers to an alkyl that is unsubstituted or substituted with one, two, or three substituents independently selected from the group consisting of halo, nitro, cyano, hydroxy, alkoxy, amino, alkylamino, dialkylamino, and optionally substituted aryl. In one embodiment, the optionally substituted alkyl is substituted with two substituents. In another embodiment, the optionally substituted alkyl is substituted with one substituent. In another embodiment, the optionally substituted alkyl is unsubstituted. Non-limiting exemplary optionally substituted alkyl groups include —CH₂Ph, —CH₂CH₂NO₂, —CH₂CH₂OH, —CH₂CH₂OCH₃, and —CH₂CH₂F.

The term “halo” as used by itself or as part of another group refers to —Cl, —F, —Br, or —I.

The term “nitro” as used by itself or as part of another group refers to —NO₂.

The term “cyano” as used by itself or as part of another group refers to —CN.

The term “hydroxy” as used by itself or as part of another group refers to —OH.

The term “amino” as used by itself or as part of another group refers to —NH₂.

The term “haloalkyl” as used by itself or as part of another group refers to an alkyl substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used by itself or as part of another group refers to an optionally substituted alkyl attached to a terminal oxygen atom. In one embodiment, the alkoxy group is a C₁₋₆ alkyl attached to a terminal oxygen atom. In another embodiment, the alkoxy group is a C₁₋₄ alkyl attached to a terminal oxygen atom. Non-limiting exemplary alkoxy groups include methoxy, ethoxy, and tert-butoxy.

The term “aryl” as used by itself or as part of another group refers to unsubstituted monocyclic or bicyclic aromatic ring systems having from six to fourteen carbon atoms, i.e., a C₆₋₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl

The term “optionally substituted aryl” as used herein by itself or as part of another group refers to an aryl that is either unsubstituted or substituted with one to five substituents independently selected from the group consisting of halo, nitro, cyano, hydroxy, alkyl, alkoxy, amino, alkylamino, dialkylamino, haloalkyl, and heterocyclo. In one embodiment, the optionally substituted aryl is an optionally substituted phenyl. In another embodiment, the optionally substituted phenyl has one substituent. In another embodiment, the optionally substituted phenyl is unsubstituted. Non-limiting exemplary substituted aryl groups include 2-methylphenyl, 2-methoxyphenyl, 2-fluorophenyl, and 4-chlorophenyl

The term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one embodiment, the cycloalkyl group has two rings. In one embodiment, the cycloalkyl group has one ring. In another embodiment, the cycloalkyl group is a C₃₋₈ cycloalkyl. In another embodiment, the cycloalkyl group is a C₃₋₆ cycloalkyl. In another embodiment, the cycloalkyl group is a C₃₋₅ cycloalkyl. The term “cycloalkyl” is meant to include groups wherein a ring —CH₂— is replaced with a —C(═O)—. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, cyclopentanone, spiro[3.3]heptane, and bicyclo[3.3.1]nonane.

The term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. In one embodiment, the optionally substituted cycloalkyl is substituted with two substituents. In another embodiment, the optionally substituted cycloalkyl is substituted with one substituent. In another embodiment, the optionally substituted cycloalkyl is unsubstituted.

The term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. The term “heterocyclo” is meant to include cyclic ureido groups such as imidazolidinyl-2-one, cyclic amide groups such as β-lactam, γ-lactam, δ-lactam and ε-lactam, and cyclic carbamate groups such as oxazolidinyl-2-one. In one embodiment, the heterocyclo group is a 4-, 5-, 6-, 7- or 8-membered cyclic group containing one ring and one or two oxygen and/or nitrogen atoms. In one embodiment, the heterocyclo group is a 5- or 6-membered cyclic group containing one ring and one or two nitrogen atoms. In one embodiment, the heterocyclo group is an 8-, 9-, 10-, 11-, or 12-membered cyclic group containing two rings and one or two nitrogen atoms. In one embodiment, the heterocyclo group is a 4- or 5-membered cyclic group containing one ring and one oxygen atom. The heterocyclo can be optionally linked to the rest of the molecule through a carbon or nitrogen atom. Non-limiting exemplary heterocyclo groups include 1,4-dioxane, 2-oxopyrrolidin-3-yl, 2-imidazolidinone, piperidinyl, morpholinyl, piperazinyl, pyrrolidinyl, 8-azabicyclo[3.2.1]octane (nortropane), 6-azaspiro[2.5]octane, 6-azaspiro[3.4]octane, indolinyl, indolinyl-2-one, and 1,3-dihydro-2H-benzo[d]imidazol-2-one.

The term “optionally substituted heterocyclo” as used herein by itself or part of another group refers to a heterocyclo that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. Non-limiting exemplary optionally substituted heterocyclo groups include:

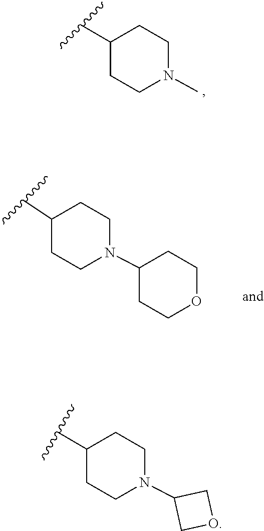

The term “(cycloalkyl)alkyl” as used by itself or as part of another group refers to an alkyl substituted with one optionally substituted cycloalkyl group. In one embodiment, the (cycloalkyl)alkyl is a C₁₋₄ alkyl substituted with one optionally substituted C₃₋₆ cycloalkyl. In one embodiment, the optionally substituted cycloalkyl group is substituted with a heterocyclo group. Non-limiting exemplary (cycloalkyl)alkyl groups include:

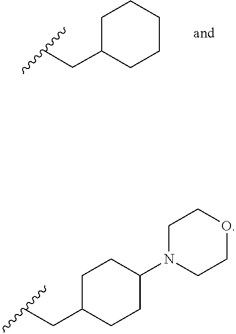 [SMILES: *CC1CCCCC1 ; *CC1CCC(N2CCOCC2)CC1]

The term “alkylamino” as used by itself or as part of another group refers to —NHR¹⁰, wherein R¹⁰ is C₁₋₆ alkyl. In one embodiment, R¹⁰ is C₁₋₄ alkyl. Non-limiting exemplary alkylamino groups include —N(H)CH₃ and —N(H)CH₂CH₃.

The term “dialkylamino” as used by itself or as part of another group refers to —NR^{11a}R^{11b}, wherein R^{11a} and R^{11b} are each independently C₁₋₆ alkyl. In one embodiment, Ru^{11a} and R^{11b} are each independently C₁₋₄ alkyl. Non-limiting exemplary dialkylamino groups include —N(CH₃)₂ and —N(CH₃)CH₂CH(CH₃)₂.

The term “(heterocyclo)alkyl” as used by itself or as part of another group refers to an alkyl substituted with one optionally substituted heterocyclo group. In one embodiment, the (heterocyclo)alkyl is a C₁₋₄ alkyl substituted with one optionally substituted 4- to 6-membered heterocyclo group. The heterocyclo can be linked to the alkyl group through a carbon or nitrogen atom. Non-limiting exemplary (heterocyclo)alkyl groups include:

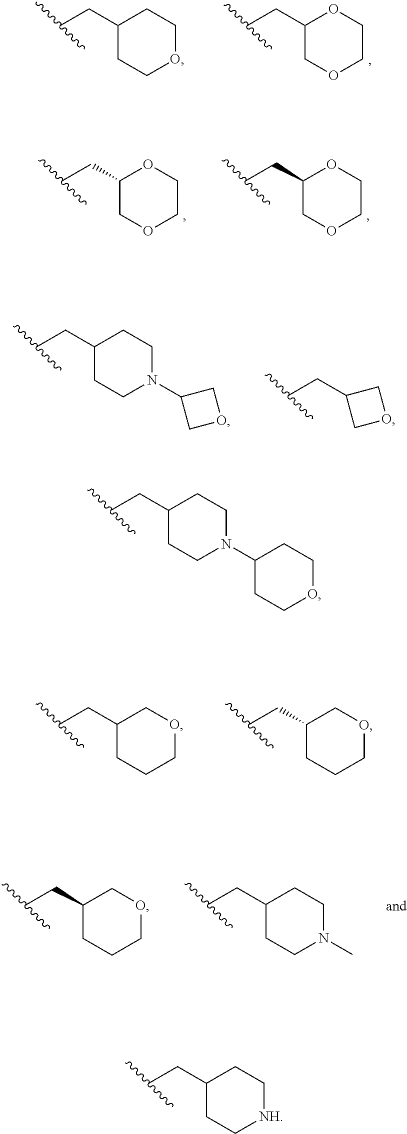

The term “heteroalkyl” as used by itself or part of another group refers to unsubstituted straight- or branched-chain aliphatic hydrocarbons containing from six to twelve chain atoms, i.e., 6- to 12-membered heteroalkyl, or the number of chain atoms designated, wherein at least two —CH₂— groups are independently replaced with —O—, —N(H)—, or —S—. The —O—, —N(H)—, or —S— can independently be placed at any interior position of the aliphatic hydrocarbon chain so long as each —O—, N(H)—, or —S— group is separated by at least two —CH₂-groups. In one embodiment, two —CH₂— groups are replaced with two —O— groups. In another embodiment, three —CH₂— groups are replaced with three —O— groups. Non-limiting exemplary heteroalkyl groups include —CH₂CH₂OCH₂CH₂OCH₃, —CH₂CH₂OCH₂CH₂N(H)CH₃, and —CH₂CH₂OCH₂CH₂OCH₂CH₂OCH₃.

The term “pharmaceutically acceptable” refers to those properties and/or substances that are acceptable to the subject to the patient from a pharmacological/toxicological point of view and to the manufacturing pharmaceutical chemist from a physical/chemical point of view regarding composition, formulation, stability, patient acceptance and bioavailability.

The term “reducing inflammation” as used herein refers to decreasing pain, redness, swelling, heat, and/or loss of function in a subject as compared to the pain, redness, swelling, heat. The reduced inflammation may be reduced neuroinflammation. Methods for assessing reduced inflammation in subjects with MS are known in the art and include, for example, Expanded Disability Status Scale (EDSS), or tracking gadolinium-enhanced brain lesions by Magnetic Resonance Imaging (MRI).

The present disclosure encompasses the preparation and use of salts of the Compounds of the Disclosure, including non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. The term “pharmaceutically acceptable salt” as used herein, refers to any salt, e.g., obtained by reaction with an acid or a base, of a Compound of the Disclosure that is physiologically tolerated in the target patient (e.g., a mammal, e.g., a human).

Acid addition salts can be formed by mixing a solution of the particular Compound of the Disclosure with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the compound of the present disclosure with a solution of a pharmaceutically acceptable non-toxic base such as sodium hydroxide, potassium hydroxide, choline hydroxide, sodium carbonate and the like.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, ethanol, and the like, and it is intended that the disclosure includes both solvated and unsolvated forms of Compounds of the Disclosure.

In one embodiment, the solvate is a hydrate. A “hydrate” relates to a particular subgroup of solvates where the solvent molecule is water. Solvates typically can function as pharmacological equivalents. Preparation of solvates is known in the art. A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

As used herein, the terms “treat”, “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective amount” as used herein refers to an amount of the active ingredient(s) that is(are) sufficient, when administered by a method of the disclosure, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; modulate protein methylation in the target cells; and/or relieve, to some extent, one or more of the symptoms associated with the cancer. To the extent the administered compound or composition prevents growth and/or kills existing cancer cells, it may be cytostatic and/or cytotoxic.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

A Compound of the Disclosure or a pharmaceutical composition or a composition comprising a Compound of the Disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In one embodiment, a Compound of the Disclosure, a pharmaceutical composition or composition comprising a Compound of the Disclosure can be administered orally. In another embodiment, a pharmaceutical composition of the present disclosure can be administered orally and is formulated into tablets, dragees, capsules, or an oral liquid preparation. In one embodiment, the oral formulation comprises extruded multiparticulates comprising the Compound of the Disclosure.

A pharmaceutical composition or a composition of the present disclosure can contain from about 0.01 to 99 percent by weight, e.g., from about 0.25 to 75 percent by weight, of a Compound of the Disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of a Compound of the Disclosure.

A pharmaceutical composition or a composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions or compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

In another embodiment, the present disclosure provides kits which comprise a Compound of the Disclosure (or a pharmaceutical composition or a composition comprising a Compound of the Disclosure) packaged in a manner that facilitates their use to practice methods of the present disclosure. In one embodiment, the kit includes a Compound of the Disclosure (or a pharmaceutical composition or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

As used herein, po refers to oral administration.

As used herein, qd refers to one a day.

Embodiment 1. A method of treating multiple sclerosis, comprising: administering an effective amount of a compound represented by Formula I below, a pharmaceutically acceptable salt or solvate thereof to a subject in need thereof:

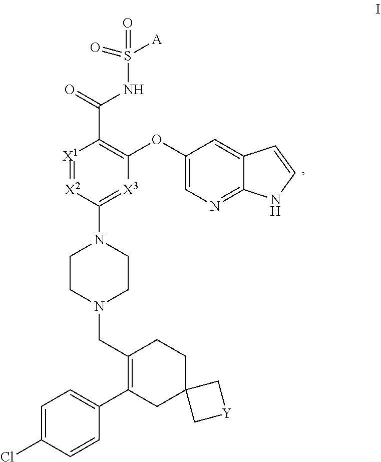

wherein:
A is

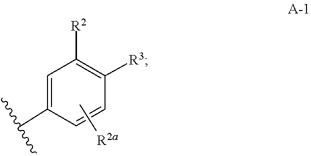

X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and
Y selected from the group consisting of —CH₂— and —O—.

Embodiment 2. The method of Embodiment 1, wherein the compound, or a pharmaceutically acceptable salt or solvate thereof, wherein X¹, X², and X³ are each —CH═;
or, X¹ is —CF═, and X² and X³ are each —CH═;
or, X¹ and X³ are each —CH═, and X² is —CF═;
or, X¹ and X² are each —CH═, and X³ is —CF═;
or, X¹ is —N═, and X² and X³ are each —CH═;
or, X¹ and X³ are each —CH═, and X² is —N═;
or, X¹ and X² are each —CH═, and X³ is —N═;
or, Y is —O—;
or, Y is —CH₂—;
or, R² is —NO₂;
or, R^{4a} is selected from the group consisting of:

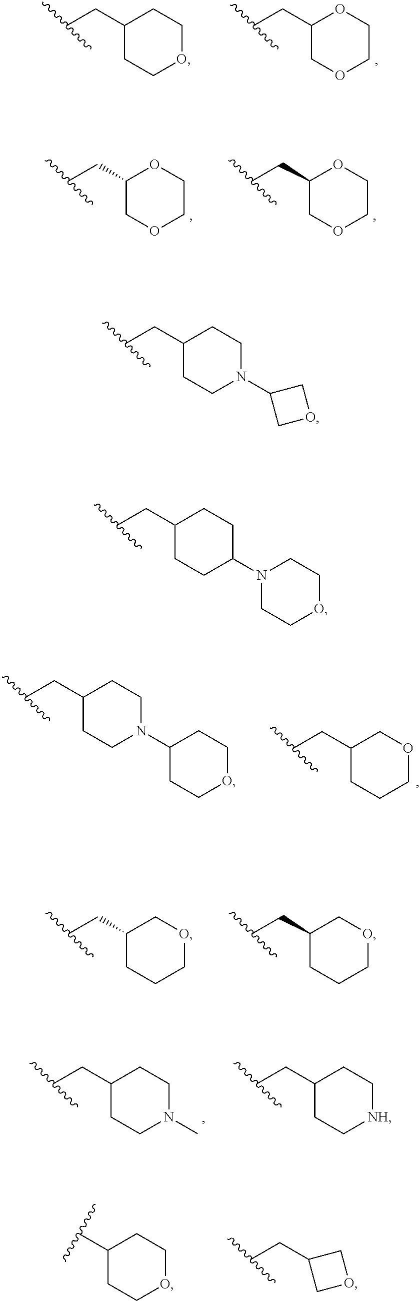

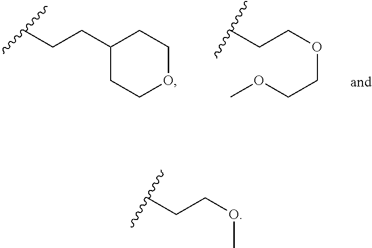

Embodiment 3. The method of Embodiment 1 or 2, wherein the compound, or a pharmaceutically acceptable salt or solvate thereof, having Formula II:

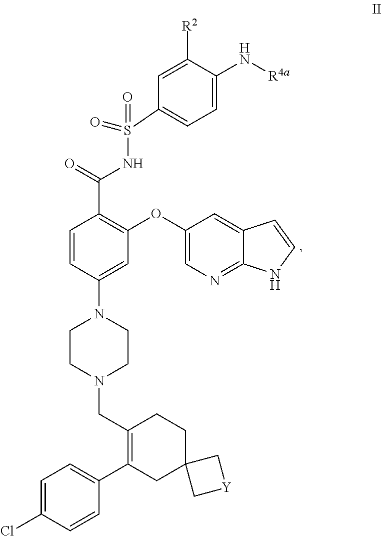

wherein Y selected from the group consisting of —CH₂— and —O—, and R² and R^{4a} are as defined in Embodiment 1 or 2.

Embodiment 4. The method of Embodiment 3, wherein the compound, or a pharmaceutically acceptable salt or solvate thereof, wherein R^{4a} is selected from the group consisting of:

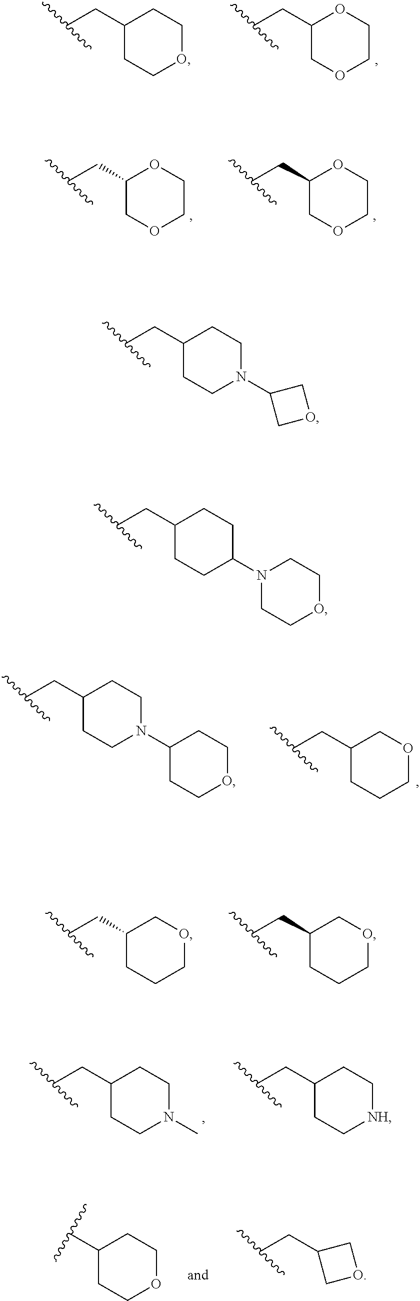

Embodiment 5. The method of Embodiment 1 or 2, wherein the compound, or a pharmaceutically acceptable salt or solvate thereof, having Formula III:

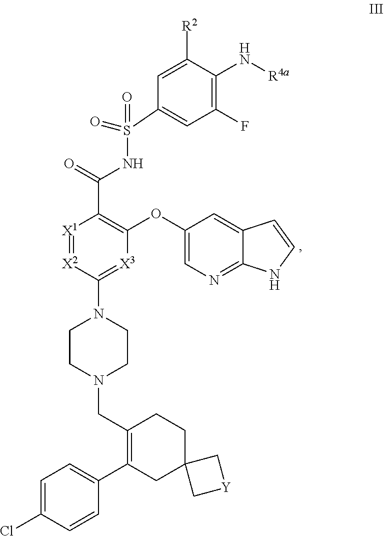

wherein Y selected from the group consisting of —CH₂— and —O—, and X¹, X², X³, R², and R^{4a} are as defined in Embodiment 1 or 2.

Embodiment 6. The method of Embodiment 1 or 2, wherein the compound, or a pharmaceutically acceptable salt or solvate thereof, having Formula IV:

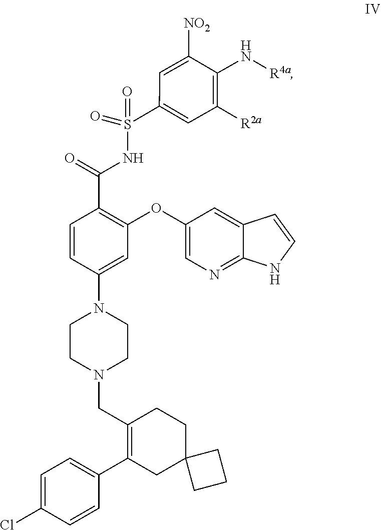

wherein R^{2a} is hydrogen or fluoro and R^{4a} is as defined in Embodiment 1 or 2.

Embodiment 7. The method of Embodiment 1, wherein the compound, or a pharmaceutically acceptable salt or solvate thereof, selected from one or more of the compounds of Table 1.

Embodiment 8. The method of Embodiment 7, wherein the compound, or a pharmaceutically acceptable salt or solvate thereof, selected from one or more of:

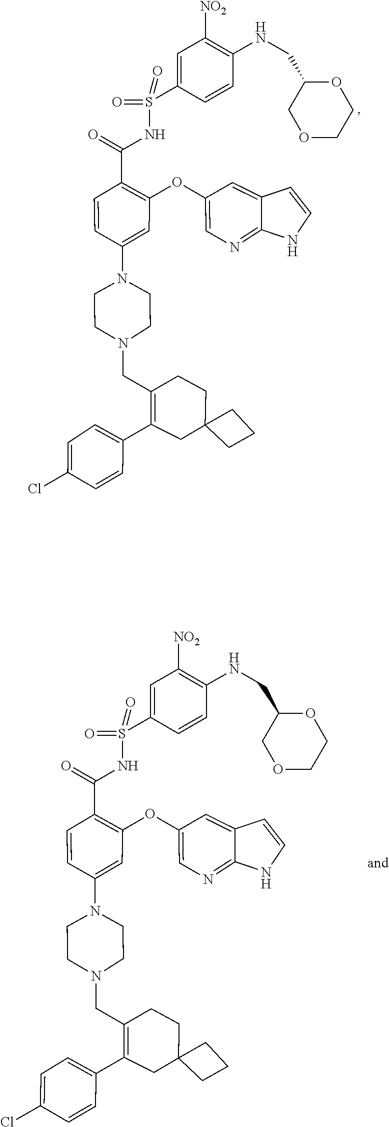 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

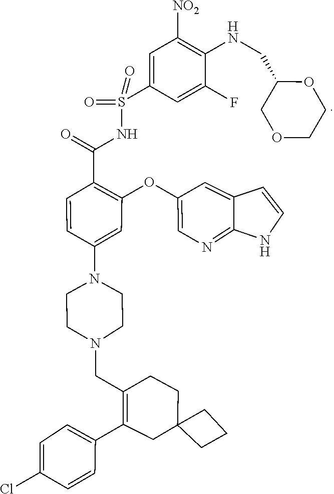 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

Embodiment 9. The method of Embodiment 8, wherein the compound, or a pharmaceutically acceptable salt or solvate thereof, which is:

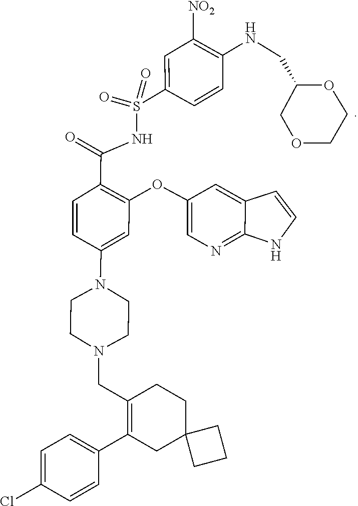 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

Embodiment 10. The method of any one of Embodiments 1 to 9, wherein the multiple sclerosis is relapsing-remitting multiple sclerosis, primary progressive multiple sclerosis or secondary progressive multiple sclerosis.

Embodiment 11. The method of any one of Embodiments 1 to 9, wherein the compound, or a pharmaceutically acceptable salt or solvate thereof is administered orally.

Embodiment 12. A method of reducing inflammation, comprising administering an effective amount of a compound as defined in any one of Embodiments 1 to 9, or a pharmaceutically acceptable salt or solvate thereof to a subject in need thereof.

Embodiment 13. The method of Embodiment 12, the inflammation is reduced in the periphery or in the central nervous system of the subject; or
the inflammation is neuroinflammation.

Embodiment 14. The method of Embodiment 12, the neuroinflammation is caused by multiple sclerosis.

Embodiment 15. A method of reducing the level of leukocytes, T lymphocytes and B lymphocytes in the PBMC in a subject, comprising administering to the subject an effective amount of a compound as defined in any one of Embodiments 1 to 9, or a pharmaceutically acceptable salt or solvate thereof, wherein the subject suffers from multiple sclerosis.

Embodiment 16. The method of Embodiment 15, the multiple sclerosis is relapsing-remitting multiple sclerosis, primary progressive multiple sclerosis or secondary progressive multiple sclerosis.

Embodiment 17. A therapeutic agent for multiple sclerosis, comprising a compound as defined in any one of Embodiments 1 to 9, or a pharmaceutically acceptable salt or solvate thereof.

Embodiment 18. A pharmaceutical composition for treating multiple sclerosis, wherein the pharmaceutical composition comprises a compound as defined in any one of Embodiments 1 to 9, or a pharmaceutically acceptable salt or solvate thereof and one or more pharmaceutically acceptable carriers.

Embodiment 19. The pharmaceutical composition of Embodiment 18, the multiple sclerosis is relapsing-remitting multiple sclerosis, primary progressive multiple sclerosis or secondary progressive multiple sclerosis.

Embodiment 20. A kit for treating multiple sclerosis, wherein the kit comprising a compound as defined in any one of Embodiments 1 to 9, or a pharmaceutically acceptable salt or solvate thereof.

Embodiment 21. The kit of Embodiment 20, the multiple sclerosis is relapsing-remitting multiple sclerosis, primary progressive multiple sclerosis or secondary progressive multiple sclerosis.

### EXAMPLES

The following examples are intended to be illustrative and are not meant in any way to limit the scope of the disclosure.

### Example 1

Experimental protocol for MOG35-55 induced experimental autoimmune encephalomyelitis (EAE) model in C57BL/6 mice

Female C57BL/6 mice (6-7 weeks old) were obtained from Shanghai Lingchang Laboratory Animal Co. Ltd.

Animals were housed and handled in a temperature-controlled environment with a 12-h light/12-h dark cycle. A total of 50 mice were assigned to 5 groups by randomization based on body weight.

The mice were anesthetized with isoflurane and immunized with 300 μg MOG 35-55 peptide emulsified (1:1) in incomplete Freund's adjuvant with 600 μg Mycobacterium tuberculosis. Each mouse received 0.05 ml emulsion (s.c.) at four sites (back shoulder and tail root). The mice were further intraperitoneally injected with 250 μL pertussis toxin (1 μg/mL) at 0 h and 48 h post immunization. The day when MOG 35-55 peptide was injected was set as day 1.

Mice in Group 1 were treated with vehicle for 20 days (po, qd, from day 1-20). Mice in Group 2, Group 3 and Group 4 were treated with 100 mg/kg Compound 4 for 20 days (po, qd, from day 1-20), 14 days (po, qd, from day 7-20) and 7 days (po, qd, from day 14-20) respectively. FTY720 was used as positive control which was orally administered to mice in Group 5 for 20 days (qd, from day 1-20). The protocols and procedures involving the care and use of animals were approved by the Institutional Animal Care and Use Committee (IACUC) at Bioduro (Shanghai, China). The information of each group is as follows.

TABLE 2

| | | Administration | Number |
|---|---|---|---|
| Group | Compound | (dosage/mpk, pathway, frequency, time/day) | of mice |
| Group 1 (G1) | Vehicle | po, qd × 20 d (d 1-d 20) | n = 10 |
| Group 2 (G2) | Compound 4 | 100 mpk, po, qd × 20 d (d 1-d 20) | n = 10 |
| Group 3 (G3) | Compound 4 | 100 mpk, po, qd × 14 d (d 7-d 20) | n = 10 |
| Group 4 (G4) | Compound 4 | 100 mpk, po, qd × 7 d (d 14-d 20) | n = 10 |
| Group 5 (G5) | FTY720 | 1 mpk, po, qd × 20 d (d 1-d 20) | n = 10 |

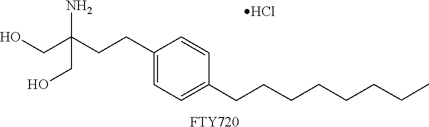

### Clinical Scores

Clinical signs of EAE were assessed every day using a scoring system from 0 to 5 based on the diameter of lymph nodes (0, normal mice, no obvious disease characteristics; 1, tail weakness or slight hind limb weakness; 2, tail weakness and hind limb weakness; 3, local hind limb weakness; 4, complete hind limb weakness; 5, on the verge of death, dead or dead due to animal welfare.)

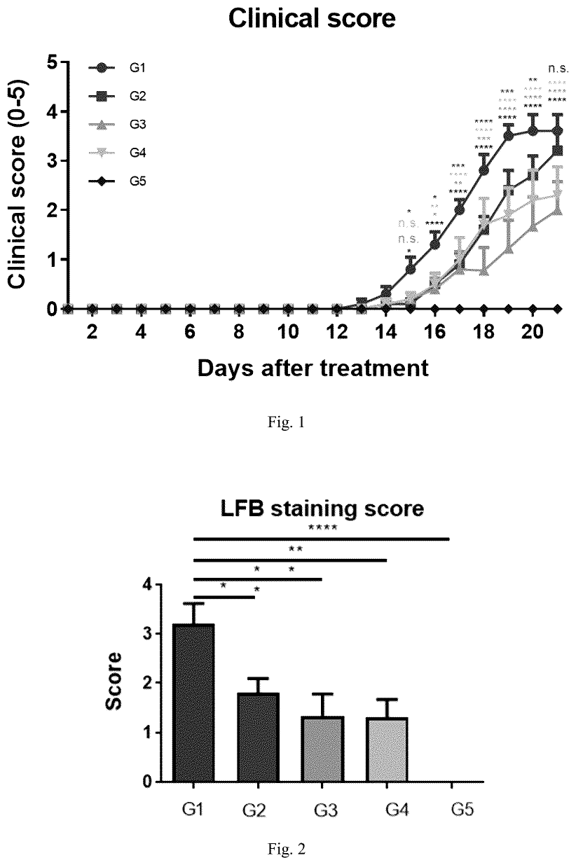

As seen in FIG. 1, mice from Group 1 (vehicle control) showed symptoms of multiple sclerosis on day 13 post immunization, and the symptoms became more severe in the following one week, reaching the maximal clinical score of 3.60±0.34 on day 20 and day 21. Treatment with FTY720 showed superior efficacy, with no mice developing symptoms of multiple sclerosis during the entire experiment. It should be noted that the dose for FTY720 was far above its clinically relevant dose, plus obvious cardiovascular toxicity observed in its clinical application. Compound 4 showed efficacy in this mouse model of multiple sclerosis by significantly reducing the clinical score of EAE when dosed at either of the three regimens. Of note, 14 days of Compound 4 administration (Group 3) appeared to be the most effective regimen in terms of clinical score, which resulted in the lowest clinical score of 2.00±0.58 at the end of the study (day 21).

### Histopathologic Assessment

At the end of the study (day 21), all the animals were sacrificed by CO₂, and the spinal cords were collected and fixed in 10% formalin for pathological analysis (H&E and LFB staining). The histopathological score was evaluated microscopically in a blinded manner.

The structure and morphology of spinal cord was evaluated by LFB staining using a scoring system from 0 to 4: 0 represents for no demyelination; 1, a small range of demyelination; 2, 2-3 small range of demyelination; 3, 1-2 large range of demyelination; 4, large-scale demyelination involves more than 20% of white matter areas.

As seen in FIG. 2, mice in the vehicle group (G1) showed a score of 3.20±0.42, indicating large range of demyelination, while Compound 4 treatment significantly reduced the score to 1.80±0.29 (p<0.05, vs vehicle), 1.33±0.44 (p<0.01, vs vehicle), 1.30±0.37 (p<0.001, vs vehicle) after 20 days, 14 days and 7 days of treatment respectively, which was in agreement with the alleviation of clinical symptoms as indicated by the clinical scores. FTY720 reduced the score of LPB staining to 0.

The inflammation of spinal cord was evaluated by H&E staining using a scoring system from 0 to 4: 0, no inflammatory cell infiltration; 1, inflammatory cell infiltration is limited to perivascular and periorbital; 2, small amount of inflammatory cell infiltration in the spinal cord; 3, medium number of inflammatory cell infiltration in the spinal cord; 4, large number of inflammatory cell infiltration in the spinal cord.

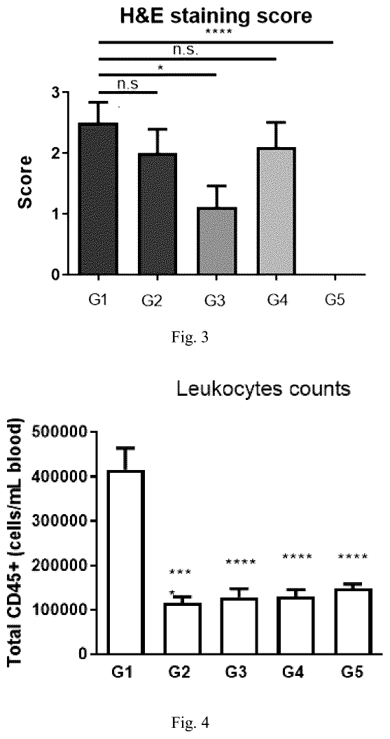

As seen in FIG. 3, mice in the vehicle group (G1) showed a score of 2.50±0.34, indicating small to medium level of inflammatory cell infiltration in the spinal cord. Compound 4 treatment reduced the score to 2.00±0.39, 1.11±0.33, 2.10±0.41 after 20 days, 14 days and 7 days of treatment respectively. It should be noted that 14 days of Compound 4 treatment most significantly reduced the H&E staining score (p<0.05, vs vehicle), which was in agreement with the most significant reduction of clinical scores at this regimen. FTY720 reduced the score of H&E staining to 0.

### Flow Cytometry

At the end of the study (day 21), peripheral blood mononuclear cell (PBMC) were obtained after lysing red blood cells in the whole blood. Cells were stained with the following florescence-labelled antibodies: APC-Cy7-conjugated anti-mouse CD45, BB700-conjugated anti-mouse CD3e, FITC-conjugated anti-mouse CD8a, Horizon V500-conjugated anti-mouse CD4 and PE-conjugated anti-mouse B220. All cells were primarily gated on live lymphocytes based on forward scatter (FCS) and side scatter (SSC). Samples were analyzed on a flow cytometer (BD FACSCalibur, USA) to count the number of each subtype of lymphocytes.

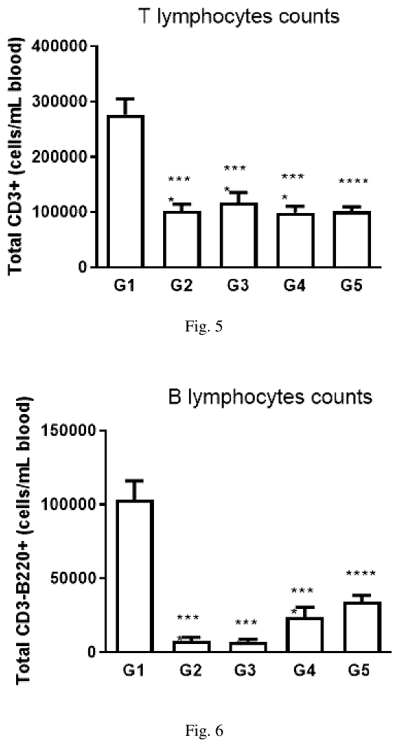

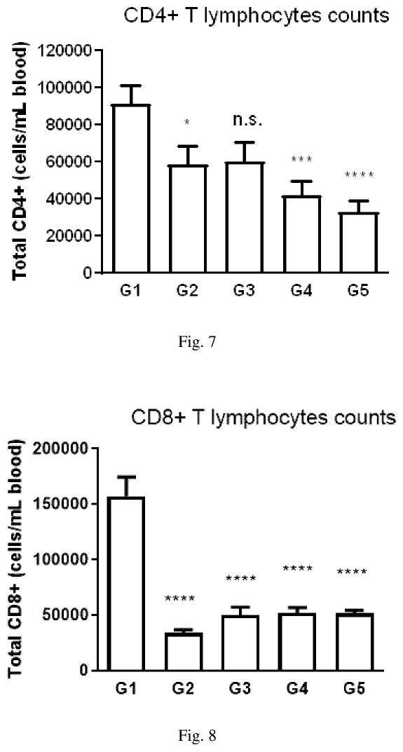

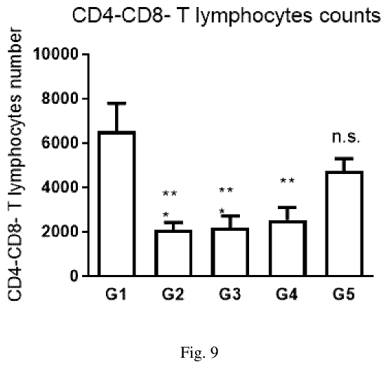

As seen in FIG. 4 to FIG. 9, after treatment with 100 mg/kg Compound 4, the numbers of leukocytes (CD45+), T lymphocytes (T cells, CD45+CD3+), B lymphocytes (B cells, CD45+CD3-B220+), CD4+ T lymphocytes (CD4+ T cells, CD45+CD3+CD4+), CD8+ T lymphocytes (CD8+ T cells, CD45+CD3+CD8+), and double negative T lymphocytes (double negative T cells, CD45+CD3+CD4-CD8−) were significantly reduced, which might explain the observed efficacy because pathological T and B cells were involved in the disease progression of multiple sclerosis.

It is to be understood that the foregoing description of two preferred embodiments is intended to be purely illustrative of the principles of the invention, rather than exhaustive thereof, and that changes and variations will be apparent to those skilled in the art, and that the present invention is not intended to be limited other than expressly set forth in the following claims.

1. A method of treating multiple sclerosis, comprising: administering an effective amount of a compound represented by Formula I below, or a pharmaceutically acceptable salt or solvate thereof to a subject in need thereof: 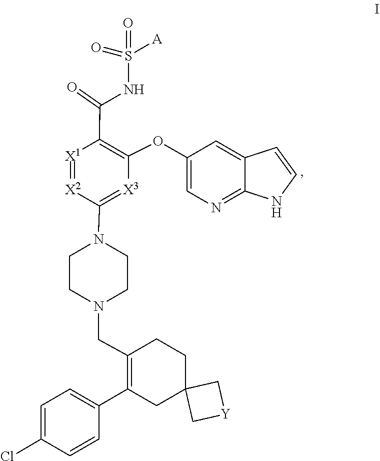
wherein:
A is 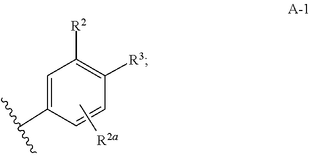
X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and
Y selected from the group consisting of —CH₂— and —O—.

2. The method of claim 1, wherein X¹, X², and X³ are each —CH═;
X¹ is —CF═, and X² and X³ are each —CH═;
X¹ and X³ are each —CH═, and X² is —CF═;
X¹ and X² are each —CH═, and X³ is —CF═;
X¹ is —N═, and X² and X³ are each —CH═;
X¹ and X³ are each —CH═, and X² is —N═; or
X¹ and X² are each —CH═, and X³ is —N═; and
Y is —O— or
—CH₂—; and
R² is —NO₂; and
R^{4a} is selected from the group consisting of:

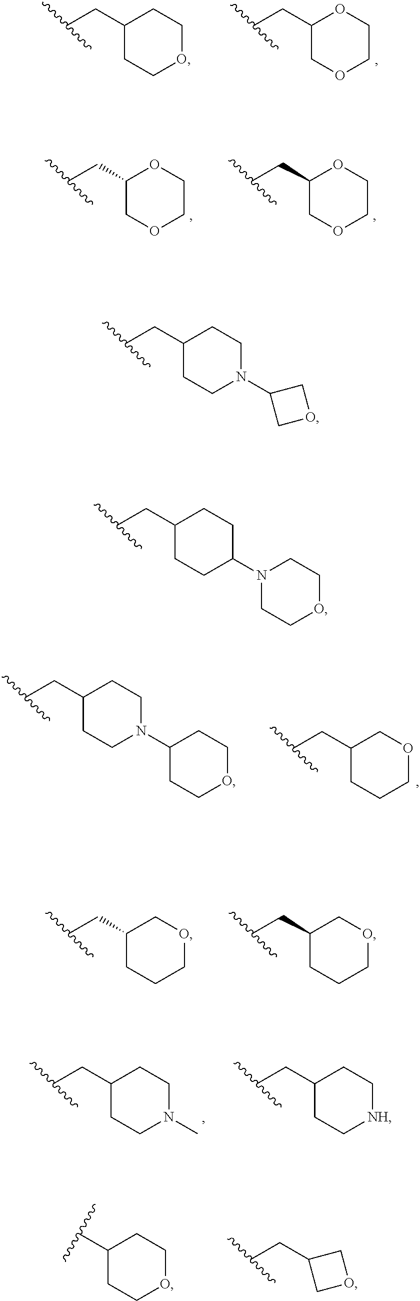

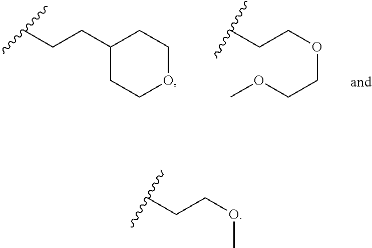

3. The method of claim 1, wherein the compound is represented by Formula II below, or a pharmaceutically acceptable salt or solvate thereof: 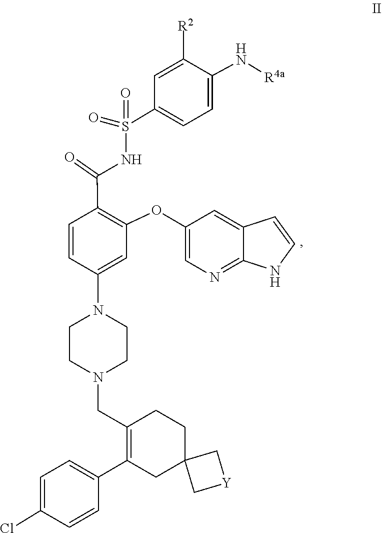
wherein Y selected from the group consisting of —CH₂— and —O—, and R² and R^{4a} are as defined in claim 1.

4. The method of claim 3, wherein R^{4a} is selected from the group consisting of:

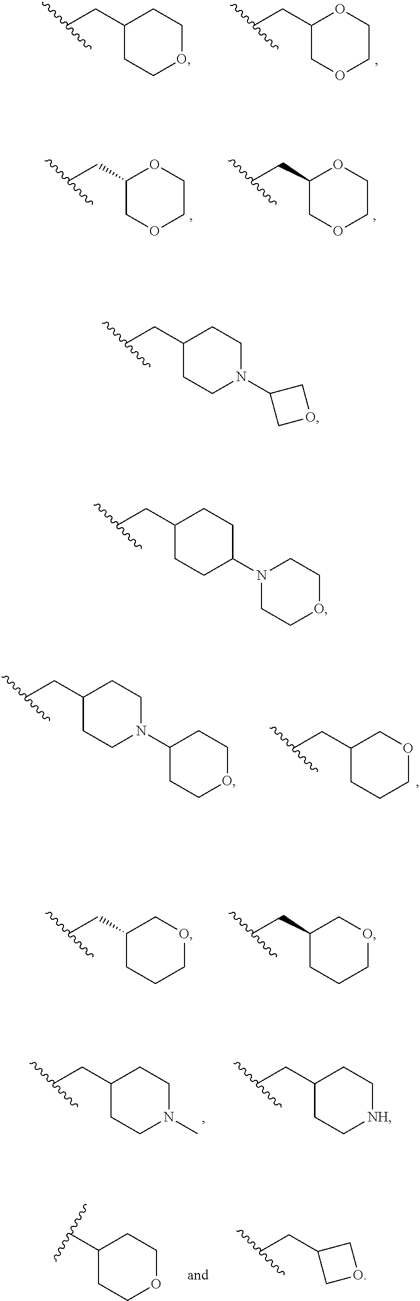

5. The method of claim 1, wherein the compound is represented by Formula III below, or a pharmaceutically acceptable salt or solvate thereof: 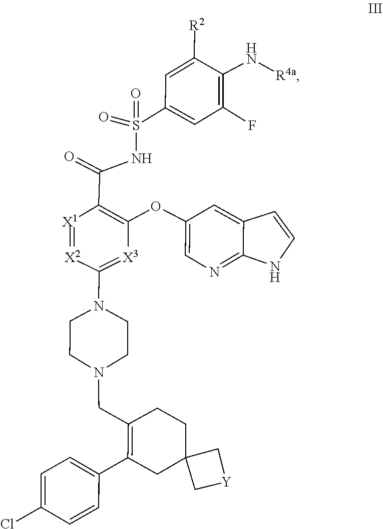
wherein Y selected from the group consisting of —CH₂— and —O—, and X¹, X², X³, R², and R^{4a} are as defined in claim 1.

6. The method of claim 1, wherein the compound is represented by Formula IV below, or a pharmaceutically acceptable salt or solvate thereof: 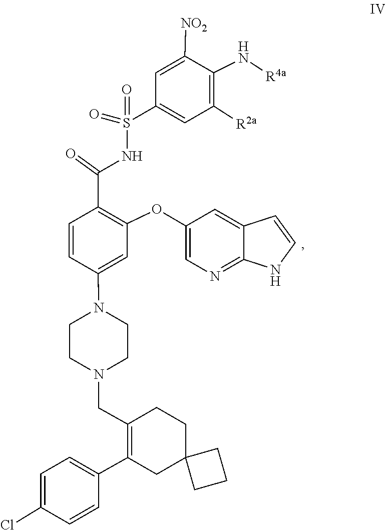
wherein R^{2a} is hydrogen or fluoro and R^{4a} is as defined in claim 1.

7. The method of claim 1, wherein the compound is selected from:

| Cpd. | | |
|---|---|---|
| No. | Structure | Name |
| 1 | 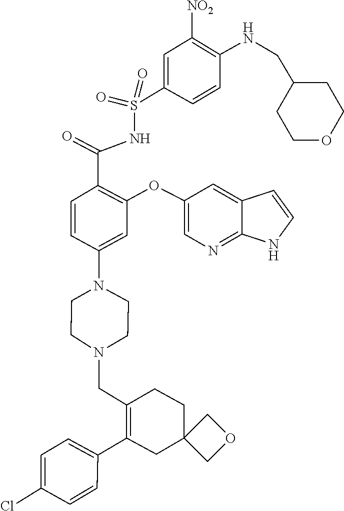 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(4- ((6-(4-chlorophenyl)-2-oxaspiro[3.5]non-6-en- 7-yl)methyl)piperazin-1-yl)-N-((3-nitro-4- (((tetrahydro-2H-pyran-4- yl)methyl)amino)phenyl)sulfonyl)benzamide |
| 2 | 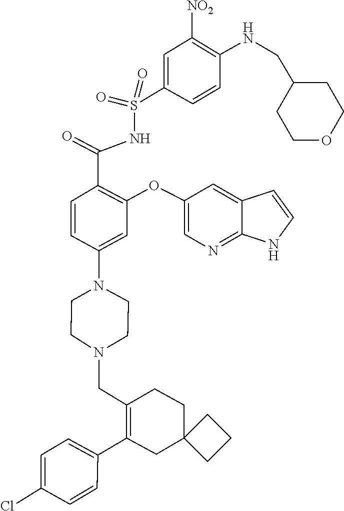 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)benzamide |
| 3 | 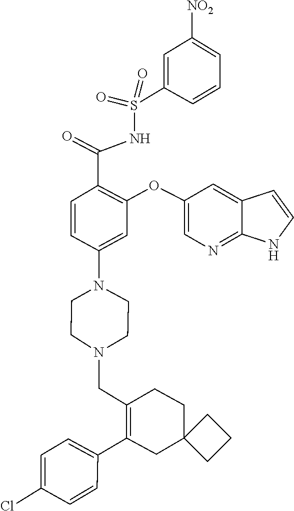 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3- nitrophenyl)sulfonyl)benzamide |
| 4 | 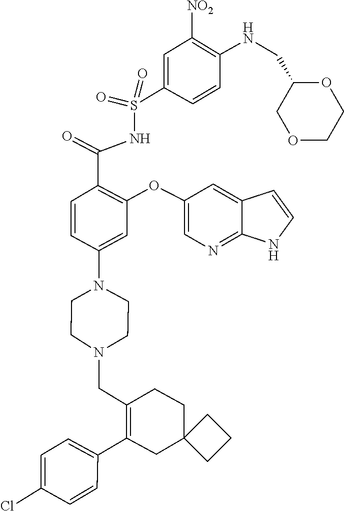 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 5 | 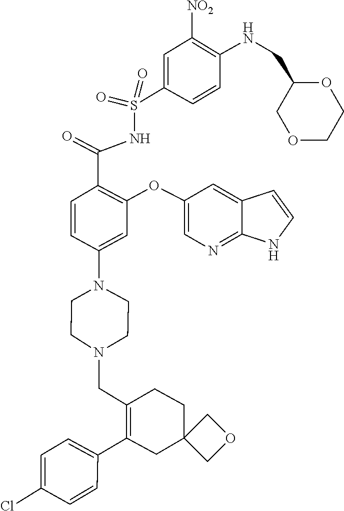 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4-chlorophenyl)- 2-oxaspiro[3.5]non-6-en-7- yl)methyl)piperazin-1-yl)benzamide |
| 6 | 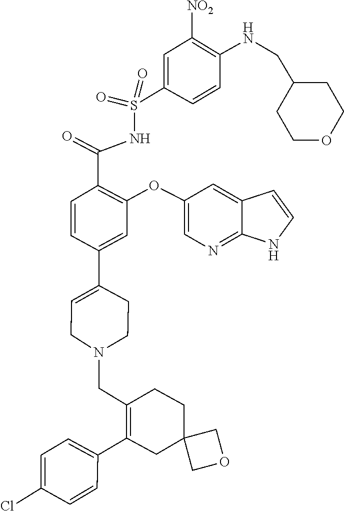 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(1- ((6-(4-chlorophenyl)-2-oxaspiro[3.5]non-6-en- 7-yl)methyl)-1,2,3,6-tetrahydropyridin-4-yl)- N-((3-nitro-4-(((tetrahydro-2H-pyran-4- yl)methyl)amino)phenyl)sulfonyl)benzamide |
| 7 | 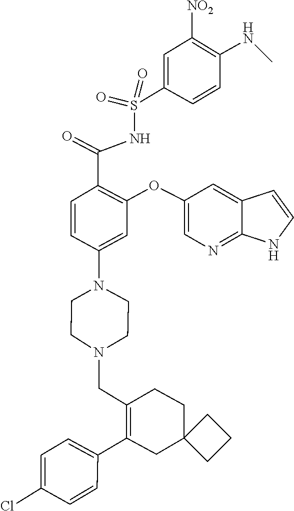 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4- (methylamino)-3- nitrophenyl)sulfonyl)benzamide |
| 8 | 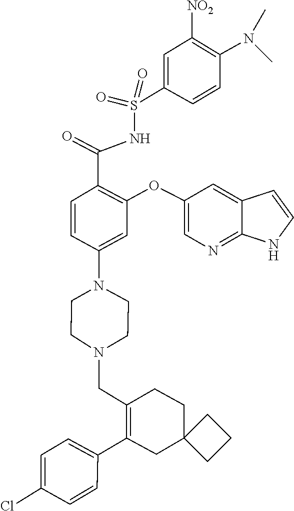 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4- (dimethylamino)-3- nitrophenyl)sulfonyl)benzamide |
| 9 | 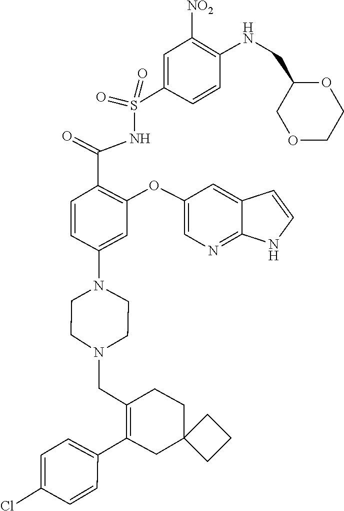 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 10 | 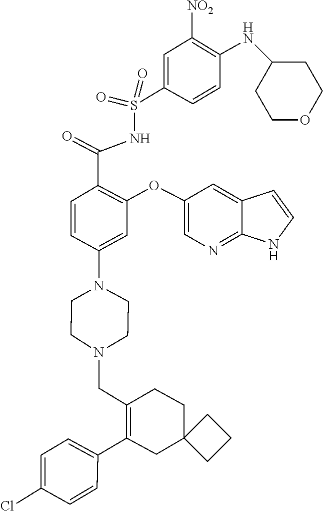 [SMILES: O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((tetrahydro-2H-pyran-4- yl)amino)phenyl)sulfonyl)benzamide |
| 11 | 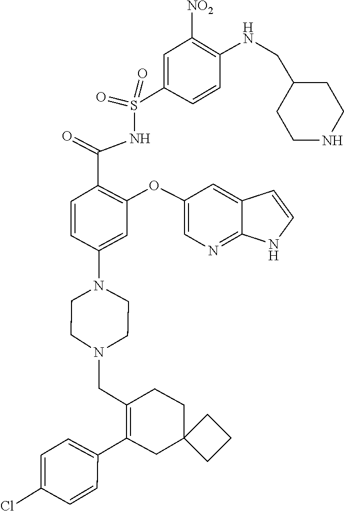 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((piperidin-4- ylmethyl)amino)phenyl)sulfonyl)benzamide |
| 12 | 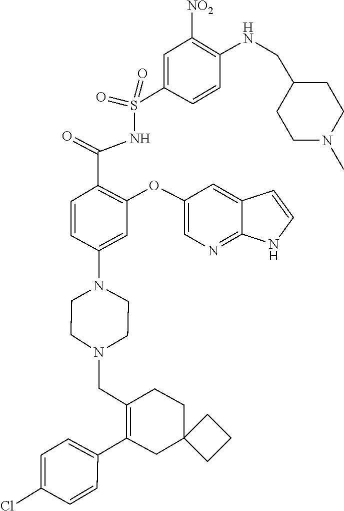 [SMILES: CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-(((1- methylpiperidin-4-yl)methyl)amino)-3- nitrophenyl)sulfonyl)benzamide |
| 13 | 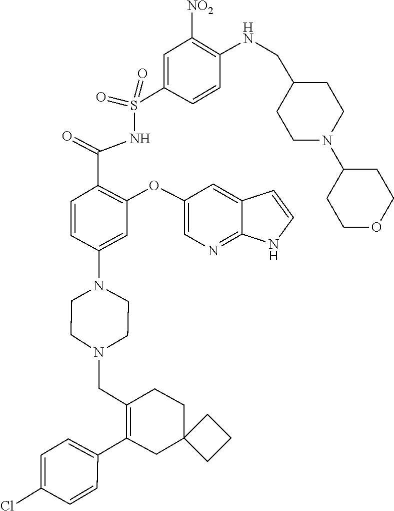 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4-(((1- (tetrahydro-2H-pyran-4-yl)piperidin-4- yl)methyl)amino)phenyl)sulfonyl)benzamide |
| 14 | 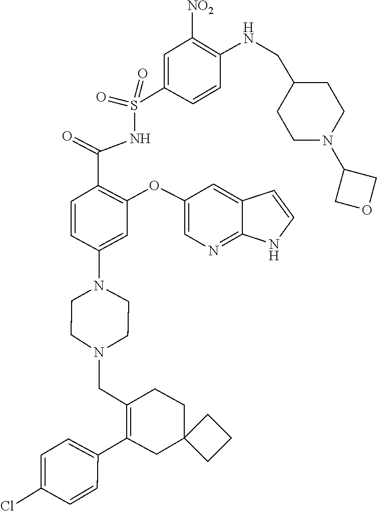 [SMILES: O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4-(((1- (oxetan-3-yl)piperidin-4- yl)methyl)amino)phenyl)sulfonyl)benzamide |
| 15 | 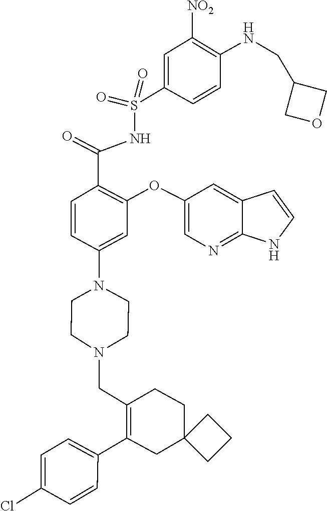 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-nitro-4- ((oxetan-3- ylmethyl)amino)phenyl)sulfonyl)benzamide |
| 16 | 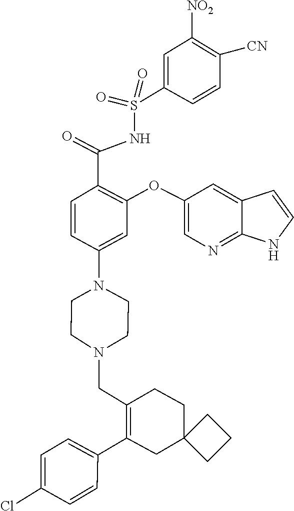 [SMILES: N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-cyano-3- nitrophenyl)sulfonyl)benzamide |
| 17 | 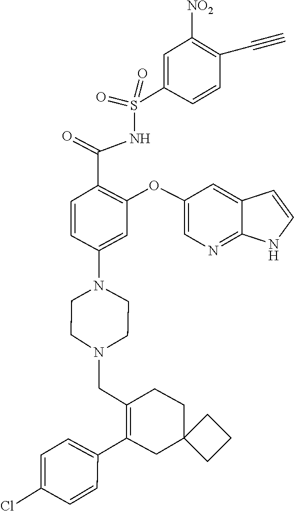 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((4-ethynyl-3- nitrophenyl)sulfonyl)benzamide |
| 18 | 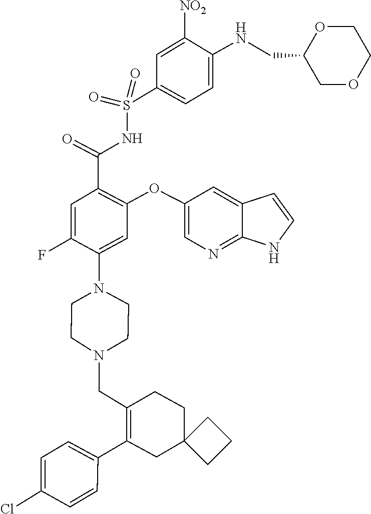 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-5-fluorobenzamide |
| 19 | 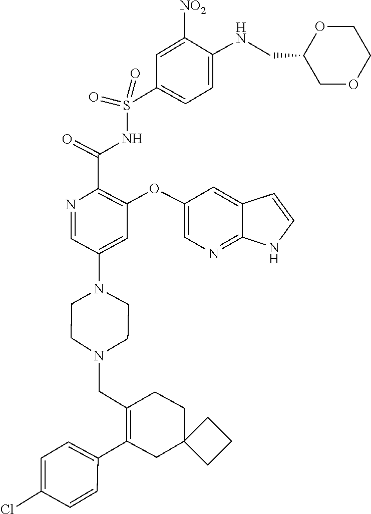 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-3-((1H-pyrrolo[2,3- b]pyridin-5-yl)oxy)-5-(4-((6-(4- chlorophenyl)spiro[3.5]non-6-en-7- yl)methyl)piperazin-1-yl)picolinamide |
| 20 | 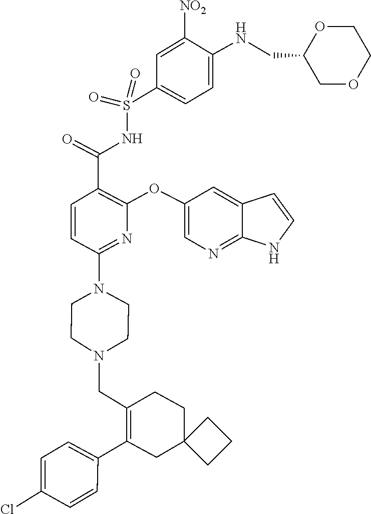 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-6-(4-((6-(4- chlorophenyl)spiro[3.5]non-6-en-7- yl)methyl)piperazin-1-yl)nicotinamide |
| 21 | 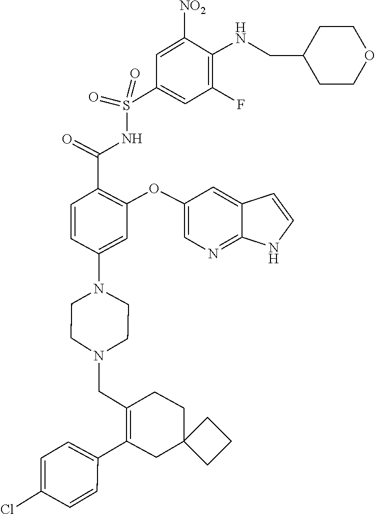 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-N-((3-fluoro-5-nitro- 4-(((tetrahydro-2H-pyran-4- yl)methyl)amino)phenyl)sulfonyl)benzamide |
| 22 | 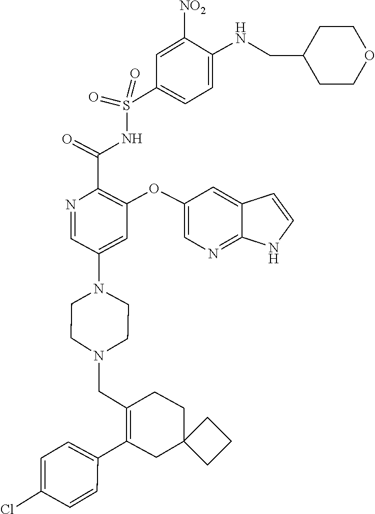 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1] | 3-((1H-pyrrolo[2,3-b]pyridin-5-yl)oxy)-5-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)picolinamide |
| 23 | 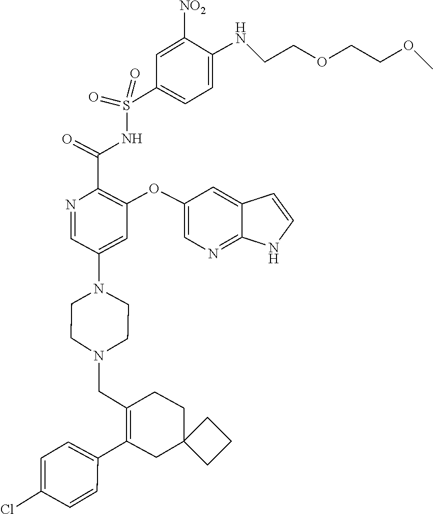 [SMILES: COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]] | 3-((1H-pyrrolo[2,3-b]pyridin-5-yl)oxy)-5-(4- ((6-(4-chlorophenyl)spiro[3.5]non-6-en-7- yl)methyl)piperazin-1-yl)-N-((4-((2-(2- methoxyethoxy)ethyl)amino)-3- nitrophenyl)sulfonyl)picolinamide |
| 24 | 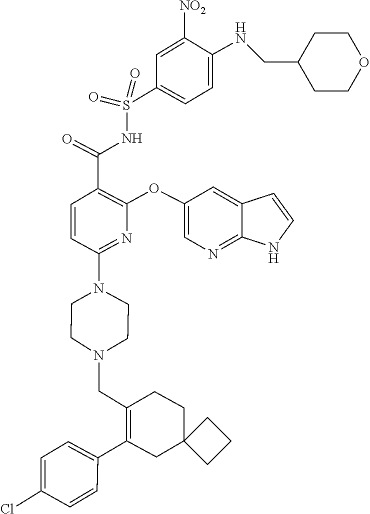 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1] | 2-((1H-pyrrolo[2,3-b]pyridin-5-yl)oxy)-6-(4- ((6-(4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)nicotinamide |
| 25 | 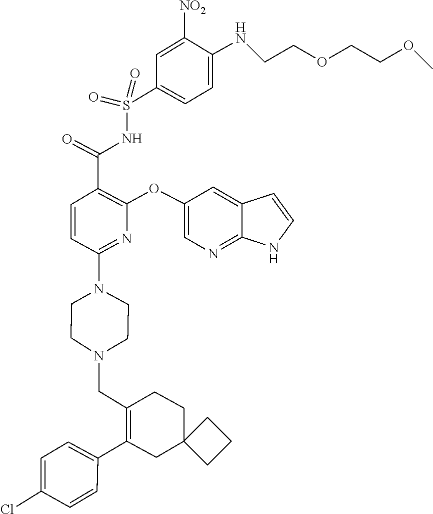 [SMILES: COCCOCCNc1ccc(S(=O)(=O)NC(=O)c2ccc(N3CCN(CC4=C(c5ccc(Cl)cc5)CC5(CCC5)CC4)CC3)nc2Oc2cnc3[nH]ccc3c2)cc1[N+](=O)[O-]] | 2-((1H-pyrrolo[2,3-b]pyridin-5-yl)oxy)-6-(4- ((6-(4-chlorophenyl)spiro[3.5]non-6-en-7- yl)methyl)piperazin-1-yl)-N-((4-((2-(2- methoxyethoxy)ethyl)amino)-3- nitrophenyl)sulfonyl)nicotinamide |
| 26 | 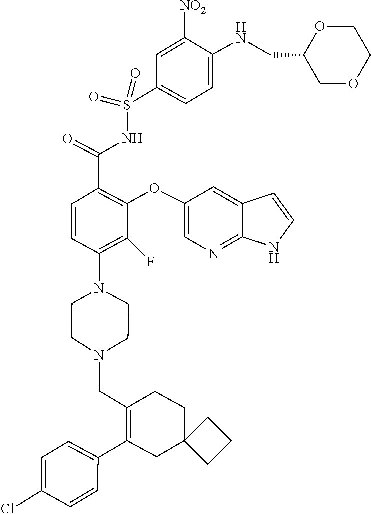 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-3-fluorobenzamide |
| 27 | 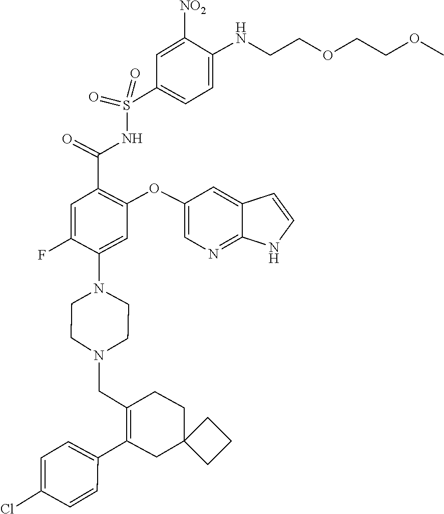 [SMILES: COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-5-fluoro-N-((4-((2- (2-methoxyethoxy)ethyl)amino)-3- nitrophenyl)sulfonyl)benzamide |
| 28 | 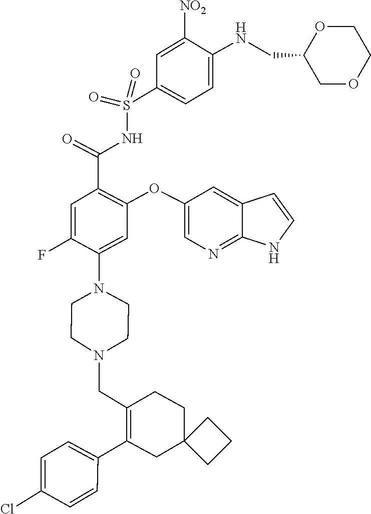 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)-5-fluorobenzamide |
| 29 | 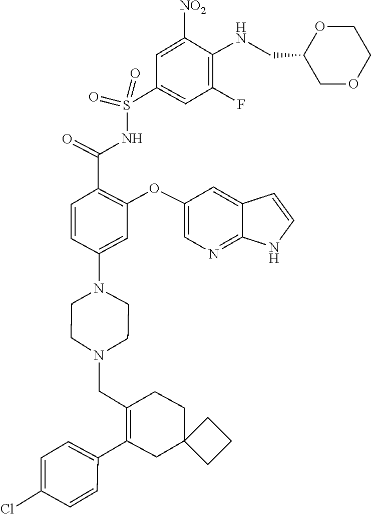 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- fluoro-5-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 30 | 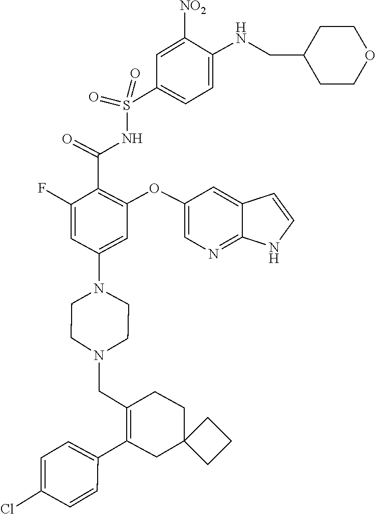 [SMILES: O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-6-fluoro-N-((3-nitro- 4-(((tetrahydro-2H-pyran-4- yl)methyl)amino)phenyl)sulfonyl)benzamide |
| 31 | 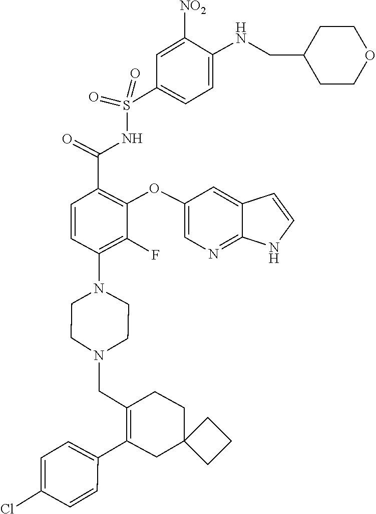 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-3-fluoro-N-((3-nitro- 4-(((tetrahydro-2H-pyran-4- yl)methyl)amino)phenyl)sulfonyl)benzamide |
| 32 | 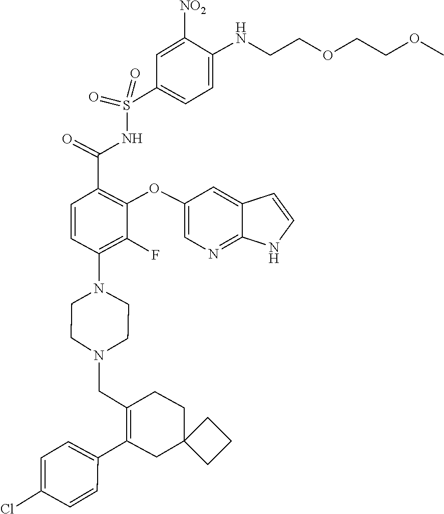 [SMILES: COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]] | 2-((1H-pyrrolo[2,3-b]pyridin-5-yl)oxy)-4-(4- ((6-(4-chlorophenyl)spiro[3.5]non-6-en-7- yl)methyl)piperazin-1-yl)-3-fluoro-N-((4-((2- (2-methoxyethoxy)ethyl)amino)-3- nitrophenyl)sulfonyl)benzamide |
| 33 | 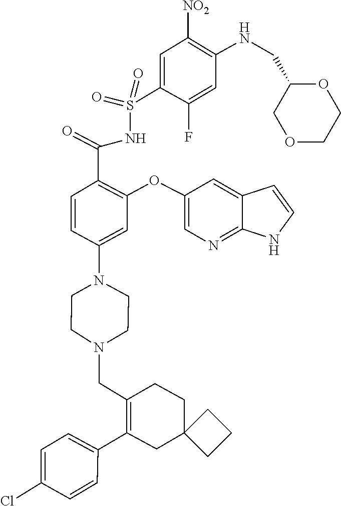 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-2- fluoro-5-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 34 | 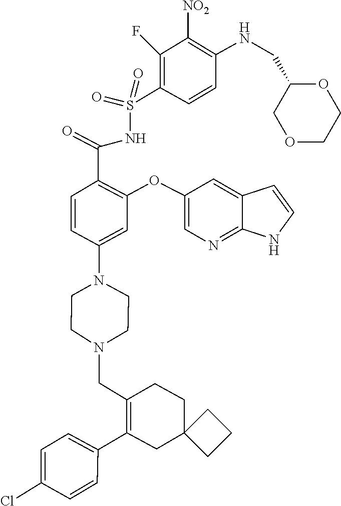 [SMILES: O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-2- fluoro-3-nitrophenyl)sulfonyl)-2-((1H- pyrrolo[2,3-b]pyridin-5-yl)oxy)-4-(4-((6-(4- chlorophenyl)spiro[3.5]non-6-en-7- yl)methyl)piperazin-1-yl)benzamide |
| 35 | 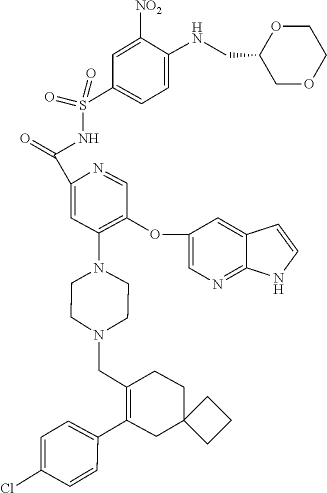 [SMILES: O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1] | (S)-N-((4-(((1,4-dioxan-2-yl)methyl)amino)-3- nitrophenyl)sulfonyl)-5-((1H-pyrrolo[2,3- b]pyridin-5-yl)oxy)-4-(4-((6-(4- chlorophenyl)spiro[3.5]non-6-en-7- yl)methyl)piperazin-1-yl)picolinamide |

or a pharmaceutically acceptable salt or solvate thereof.

8. The method of claim 7, wherein the compound is selected from the group consisting of: 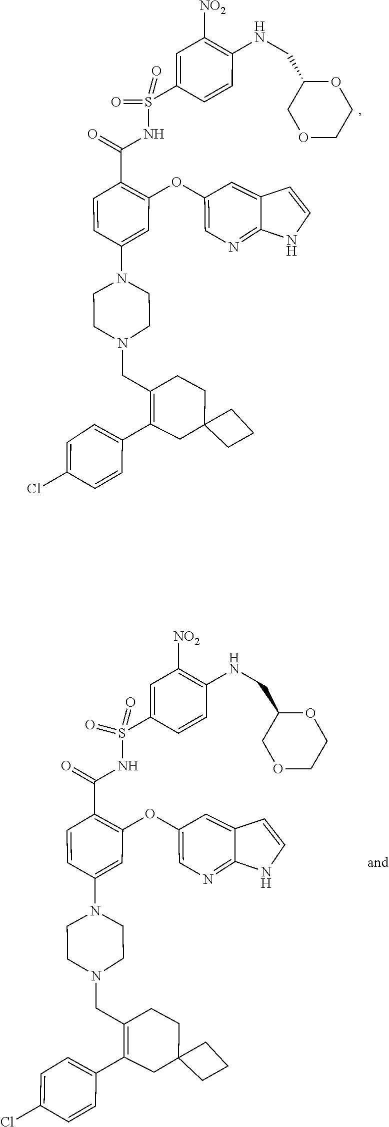 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] 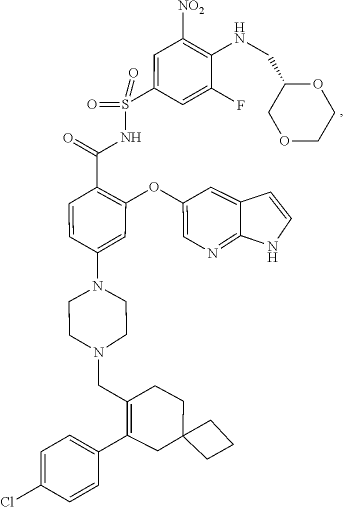 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt or solvate thereof.

9. The method of claim 8, wherein the compound is: 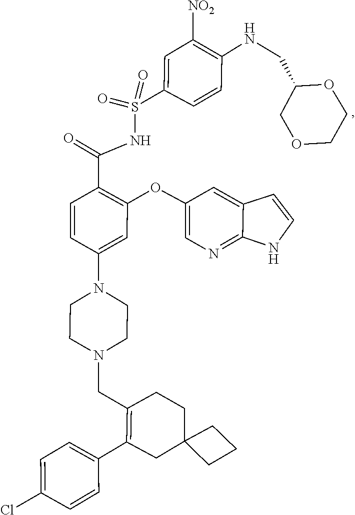 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt or solvate thereof.

10. The method of claim 1, wherein the multiple sclerosis is relapsing-remitting multiple sclerosis, primary progressive multiple sclerosis, or secondary progressive multiple sclerosis.

11. The method of claim 1, wherein the compound, or a pharmaceutically acceptable salt or solvate thereof is administered orally.

12. A method of reducing inflammation, comprising administering an effective amount of a compound as defined in claim 1, or a pharmaceutically acceptable salt or solvate thereof to a subject in need thereof.

13. The method of claim 12, wherein
the inflammation is neuroinflammation.

14. The method of claim 13, wherein the neuroinflammation is caused by multiple sclerosis.

15. The method of claim 12, wherein the method reduces the level of leukocytes, T lymphocytes and B lymphocytes in the peripheral blood mononuclear cells (PBMCs), wherein the subject suffers from multiple sclerosis.

16. The method of claim 15, wherein the multiple sclerosis is relapsing-remitting multiple sclerosis, primary progressive multiple sclerosis, or secondary progressive multiple sclerosis.

17. (canceled)

18. A pharmaceutical composition for treating multiple sclerosis, wherein the pharmaceutical composition comprises a compound as defined in claim 1, or a pharmaceutically acceptable salt or solvate thereof, and one or more pharmaceutically acceptable carriers.

19. The pharmaceutical composition of claim 18, wherein the multiple sclerosis is relapsing-remitting multiple sclerosis, primary progressive multiple sclerosis, or secondary progressive multiple sclerosis.

20-21. (canceled)

22. The method of claim 12, wherein the inflammation is reduced in the periphery or in the central nervous system of the subject.
